(19) United States
(12) Patent Application Publication
(10) Pub. No.: US 2015/0250470 A1
(43) Pub. Date: Sep. 10, 2015

(54) SUTURE ANCHORING SYSTEM AND DELIVERY METHOD

(71) Applicant: Coaxis Medical, Inc., Redwood City, CA (US)

(72) Inventor: Jaime S. Vargas, Redwood City, CA (US)

(21) Appl. No.: 14/639,669

(22) Filed: Mar. 5, 2015

Related U.S. Application Data
(60) Provisional application No. 61/948,030, filed on Mar. 5, 2014.

(51) Int. Cl. A61B 17/04

(52) U.S. Cl. CPC A61B 17/0401; A61B 2017/0409

(57) ABSTRACT

Systems and methods for anchoring sutures in various limited access surgical procedures utilize suture anchors having a displaceable section for selectively locking a portion of suture to the suture anchor. A series of anchors and optional suture locks may be used in running or interrupted stitch patterns, depending on the application.

### CROSS-REFERENCE TO RELATED APPLICATION

This application claims the benefit of priority to U.S. Provisional Patent Application No. 61/948,030, filed on Mar. 5, 2014, which is incorporated in its entirety herein by reference.

### FIELD OF THE INVENTION

The present invention, in various embodiments, relates to devices, methods, and systems for anchoring surgical sutures and, more specifically, to selectively-lockable suture anchors.

### BACKGROUND OF THE INVENTION

Suture anchors may be employed in several useful clinical applications that include, for the purpose of illustration and not limitation, flexible endoscopy, laparoscopic and robotic surgery, orthopedic surgery, skin closure, and percutaneous image-guided procedures. Suture anchors may also be used to attach various medical devices, e.g., a stent, physiologic sensor, wireless camera, or surgical mesh, to a specific anatomic location, e.g., the gastrointestinal (GI) tract. Advantageously, with suture anchoring, suture may be endoscopically or laparoscopically cut to facilitate device removal or use of absorbable type sutures could allow for delayed, spontaneous migration.

For example, with GI endoscopy, suture anchors may be used to approximate tissue layers, sections of tissue within a single tissue layer, or the walls of adjacent lumens. Tissue approximation with anchors may also facilitate several types of percutaneous medical procedures, e.g., feeding tube placement or drainage procedures. For percutaneous feeding tube placement, anchors with running suture may be placed percutaneously into a lumen, e.g., of the stomach or small intestine, and, subsequently, the suture may be tightened and the anchor locked to approximate that organ, e.g., against the abdominal wall. This practice may enable safe percutaneous feeding tube insertion directly into, e.g., the stomach or jejunum. In a similar manner, tissue anchors placed into the colon or gallbladder may facilitate direct insertion of drainage tubes in the setting of obstruction.

Suture anchors may also approximate two separate lumens to facilitate creation of an internal fistula. For example, suture anchors placed from the stomach into a loop of jejunum can be used to bring these two organs in closer proximity to facilitate the creation of a gastrojejunostomy to bypass a gastric outlet obstruction or as part of a malabsorptive bariatric procedure. Suture anchors placed from the duodenum or stomach into the biliary tree or gallbladder may also help create bilioenteric anastomoses to relieve biliary pressure and abdominal pain.

Suture anchors may also be used to repair a hiatal hernia to relieve symptoms of gastro-esophageal reflux disease by delivering the suture anchors through the esophageal wall and diaphragm muscle to prevent relative motion. Suture anchors may also be delivered through the esophageal wall, diaphragm muscle, and gastric fundus for a repair approximating a partial fundoplication.

Additionally, anchors may be inserted around a GI tract perforation, with subsequent tightening of the attached suture to close the defect. In GI endoscopy, however, current configurations of suture anchors have several limitations. For example, depending on the specific model, these limitations include: (1) need for endoscope removal and reinsertion for placement of each individual anchor, (2) ability to perform only interrupted suture placement, (3) ability to place only one set of running sutures, (4) need for a separate suture tightening/locking device, (5) need for a separate suture cutting mechanism, and/or (6) inability to place additional sutures with the same device after cutting suture. Some devices for placing endoscopic sutures involve suture delivery mechanisms attached to the outside of the endoscope; however, these devices can be quite cumbersome and difficult to use depending on the anatomic location.

With laparoscopic or robotic surgery, suture anchors with a running suture provide a more efficient method for surgical suturing as compared to current minimally invasive techniques that mimic manual tying of surgical knots. For example, current laparoscopic and robotic surgical techniques for suturing involve suture needles that are manipulated at the end of tools placed via surgical ports. Current techniques, however, usually require two access ports for performing this task and mimic what a surgeon may do using two hands. More specifically, a first access port may be used for introducing tools, e.g., to grasp the needle with suture, and a second access port may be used for tools, e.g., to manipulate the tissue that requires suturing. Once the suture has been placed, a surgical knot may be tied to cinch the suture and hold the suture in place. Because suture anchor delivery needles may be of sufficiently small diameter for port-less percutaneous insertion into the abdominal cavity, significant improved surgical access may be achieved through simple repositioning of the delivery needles. Similarly, suture anchors placed through tissues percutaneously may be used to temporarily retract internal organs to aid in laparoscopic access and visualization without requiring additional laparoscopic ports.

With orthopedic surgery, tissue anchors with running suture may provide a means of approximating two damaged tissues, e.g., a torn ligament, tendon or meniscus, or facilitate attaching a surgical implant to desired tissue. Indeed, tissue anchors with running suture may provide a very efficient and safe means for closure of cutaneous defects. Current methods for skin closure with a running stitch, however, generally involve piercing a needle with attached suture back and forth across tissue planes followed by tying a surgical knot and suture cutting. Interrupted sutures for skin closure are even more involved, as knot tying and suture cutting may be required with each interrupted stitch.

### BRIEF SUMMARY OF THE INVENTION

In view of the shortcomings of current surgical techniques and devices, it is desirable to provide a single platform device for suture anchoring, suturing, and suture cutting. More specifically, it is desirable to provide a single platform device that enables creating interrupted or running suture, and which provides several advantages over current skin closure techniques. More particularly, it is desirable to provide devices, systems, and methods that address the current limitations of suture anchors and the unmet clinical needs for a robust and easy-to-use suturing platform in a very compact operating volume.

According to one embodiment, the system described herein contains a series of anchors with suture extending slidably throughout, aligned coaxially and/or in parallel and disposed in a small diameter delivery system that may fit within the instrument channel or lumen of an endoscope or echoendoscope. The small diameter of the delivery device may facilitate its use alongside other laparoscopic and robotic surgical tools via a single surgical port and/or may also be used directly percutaneously or for cutaneous applications without a separate surgical port. The unique configurations of the suture anchors according to various embodiments and the associated delivery system enable locking of the suture on one or more desired anchors, suture cutting, and the ability to initiate further suture placement with the same device. These innovative characteristics allow for multiple suture anchor placements and the ability to place numerous interrupted or running sutures without the need for delivery device removal.

In a first aspect, a suture anchoring system is provided. In some embodiments, the system includes an elongate anchor having a longitudinal axis extending from a proximal end to a distal end of the anchor and a displaceable section, e.g., a suture loop, a suture locking tab, a deformable connecting element, a suture engagement slot, a suture engagement tooth, a deflectable locking prong, a bridge element, a deformable strut, or a cantilevered tab for locking a portion of suture so that a length of the locked portion of suture extends in a direction of the longitudinal axis. In some variations, the system may further include the suture. The anchor and/or suture may be made from a bio-resorbable material. In various embodiments, the displaceable section is made of a plastically deformable material and/or an elastically deformable material.

In some implementations, the anchor may further include a fixed section having a feature(s), e.g., a suture engagement slot, a suture engagement tooth, or a fixed tab, for engaging the suture. In some variations, the suture anchor may further include one or more of a plurality of tissue supports, an anti-rotation key mateable with a corresponding anti-rotation key of another anchor, a beveled distal tip, a sharp distal tip, a blunt shoulder proximate the distal tip, and/or a connector interlockable with a corresponding connector of another anchor to permit rotation of the anchor in a single plane. In some variations, the anchor may define a lumen for slidably accepting the suture and/or the anchor may define a suture pocket(s) for accepting a portion of the suture.

In a second aspect, a system for delivering suture anchors is provided. In some embodiments, the system includes a delivery needle and an anvil coupled to the delivery needle for locking, within the delivery needle, an anchor to a suture. In one variation, the anchor may include disengagement shoulders at proximal and distal ends of the anchor. In some implementations, the system may further include one or more anchors positioned within the delivery needle, the suture extending slidably through the anchors, a frangible connector between each anchor, a pushrod for advancing the plurality of anchors through the delivery needle, a mechanism for retracting, tensioning, and/or releasing the suture, and/or a locking tube for actuating the anvil and/or for cutting suture, e.g., using a cutting edge moveable relative to the delivery needle. In some variations, the delivery needle, each anchor, and the suture may be aligned substantially coaxially. In some implementations, the delivery needle may define a longitudinal slot for engaging a protruding tab of an anchor and/or the delivery needle may include a sharp distal tip and/or a suture-cutting notch. In one variation, the pushrod includes an anti-rotation key mateable with a corresponding anti-rotation key of an anchor.

In a third aspect, a method for suturing tissue is taught. In some embodiments, the method includes using an anvil to lock, within a delivery needle, an anchor to a suture, and deploying the anchor from the delivery needle to a tissue site. The step of using the anvil may include actuating the anvil by displacing a locking tube relative to the anvil and/or causing the anvil to mechanically interfere with a displaceable section of the anchor (e.g., by displacing the anchor with a pushrod). In some implementations, the method may further include one or more of displacing a locking tube (e.g., moving the locking tube longitudinally along a longitudinal axis of the deliver needle and/or rotating the locking tube) to cut the suture, and using the anchor and the suture to perform at least one of an approximation of tissue layers, an approximation of two separate lumens within a patient's body, a closure of a defect at the tissue site, and securing of a medical device, e.g., a stent, a physiological sensor, a camera, a surgical mesh, or the like, at the tissue site. In some variations, the method may further include advancing the delivery needle through an instrument channel of an endoscope to the tissue site.

In a fourth aspect, a method for engaging tissue is taught. In some embodiments, the method includes inserting a tissue engagement tool proximate a tissue site, with the tissue engagement tool including a catheter and a helical corkscrew element coupled to a distal end of the catheter; rotating the helical corkscrew element to engage the tissue site; and, thereafter, delivering to the tissue site a medical instrument, e.g., a tissue approximation device, a tissue aspiration needle, biopsy forceps, an injection needle, a cryotherapy probe, and an RF ablation probe, through a lumen of the catheter. In some variations, the method further includes cutting suture using a cutting edge coupled to the distal end of the catheter.

In a fifth aspect, a tissue engagement tool is provided. In some embodiments, the tool includes a catheter having a lumen for receiving a medical instrument, a cutting edge, e.g., an edge beveled radially inwards, coupled to a distal end of the catheter, and a helical corkscrew element coupled to the distal end of the catheter. In one variation, the helical corkscrew element includes a sharp distal tip for engaging tissue.

It is to be understood that the features of the various embodiments described herein are not mutually exclusive and can exist in various combinations and permutations.

### BRIEF DESCRIPTION OF THE DRAWINGS

Further features, embodiments, and advantages of the present invention will become apparent from the following detailed description with reference to the drawings, in which:

FIG. 1A shows a perspective view of an illustrative embodiment of a suture anchor in accordance with the present invention;

FIG. 1B shows a cross-sectional view of a first embodiment of a selectively lockable suture anchor with a locking tab and a fixed tab in accordance with the present invention;

FIG. 1C shows a cross-sectional view of the selectively lockable suture anchor of FIG. 1B after an anvil has pinched the locking tab in accordance with the present invention;

FIG. 1D shows a perspective view of a second embodiment of a selectively lockable suture anchor having a fixed section, a movable section, and a deformable connecting element in accordance with the present invention;

FIG. 1E shows a perspective view of a third embodiment of a selectively lockable suture anchor having a locking mechanism in accordance with the present invention;

FIG. 1F shows a perspective view of a fourth embodiment of a selectively lockable suture anchor having a locking mechanism in accordance with the present invention;

FIG. 2A shows a perspective view of a selectively lockable suture anchor proximate a delivery needle tip of a delivery and deployment system in accordance with an embodiment of the present invention;

FIG. 2B shows a cross-sectional view of selectively lockable suture anchors disposed in a delivery needle in accordance with an embodiment of the present invention;

FIG. 2C shows a cross-sectional view of the selectively lockable suture anchors and delivery needle of FIG. 2B in which the bridge elements have overridden the anvil in accordance with that embodiment of the present invention;

FIG. 2D shows a cross-sectional view of the selectively lockable suture anchor and delivery needle of FIG. 2B in which the locking tube has been advanced to force the anvil into the bridge elements in accordance with that embodiment of the present invention;

FIG. 2E shows a cross-sectional view of the selectively lockable suture anchors and delivery needle of FIG. 2D with the locking tube withdrawn in accordance with one embodiment of the present invention;

FIG. 2F shows a perspective view of an illustrative embodiment of a needle delivery system for delivering the suture anchors of FIG. 3G in accordance with the present invention;

FIG. 2G shows a perspective view of the needle delivery system of FIG. 2F deploying the suture anchor of FIG. 3G in accordance with one embodiment of the present invention;

FIG. 2H shows a perspective view of the needle delivery system of FIG. 2G rotating the suture anchor of FIG. 3G in accordance with one embodiment of the present invention;

FIG. 2I shows a cross-sectional view of an illustrative embodiment of a needle delivery system for delivering a pair of anchors locked to a connecting section of fixed length suture in accordance with the present invention;

FIG. 2J shows a cross-sectional view of the pair of anchors of FIG. 2I approximating tissue lumen walls in accordance with one embodiment of the present invention;

FIG. 3A shows a cross-sectional view of an illustrative embodiment of a double wiggle anchoring mechanism for a suture anchor in accordance with the present invention;

FIG. 3B shows top and bottom perspective views of an illustrative embodiment of a double wiggle suture anchor in accordance with the present invention;

FIG. 3C shows a perspective view of an illustrative embodiment of a suture anchor having a cylinder-plane snap lock in accordance with the present invention;

FIG. 3D shows a cross-section of the snap lock of FIG. 3C in accordance with the present invention;

FIG. 3E shows a perspective view of an illustrative embodiment of a suture anchor having angled bridge elements and two lower suture pockets in accordance with the present invention;

FIG. 3F shows a perspective view of an illustrative embodiment of a suture anchor having angled struts leading to a single bridge and a single lower suture pocket in accordance with the present invention;

FIG. 3G shows a perspective view of an illustrative embodiment of a suture anchor having at least one cantilevered tab in accordance with the present invention;

FIG. 4 shows a perspective view of an illustrative embodiment of a sharpened-tip suture anchor in accordance with the present invention;

FIG. 5A shows a top perspective view of an illustrative embodiment of a bridge and teeth-type suture anchor in accordance with the present invention;

FIG. 5B shows a bottom perspective view of the bridge and teeth-type suture anchor of FIG. 5A in accordance with the present invention;

FIG. 6A shows a perspective view of an illustrative embodiment of a suture anchor with an alignment tab in accordance with the present invention;

FIG. 6B shows a perspective view of an illustrative embodiment of a delivery needle having an alignment slot for use with the alignable suture anchor of FIG. 6A in accordance with the present invention;

FIG. 7A shows a perspective view of an illustrative embodiment of multiple suture anchors having beveled interlocking links in accordance with the present invention;

FIG. 7B shows an enlarged perspective view of an illustrative embodiment of the interaction between the beveled interlocking links of the suture anchors in FIG. 7A in accordance with the present invention;

FIG. 8A shows a perspective view of an illustrative embodiment of multiple suture anchors having frangible connectors in accordance with the present invention;

FIG. 8B shows an enlarged top perspective view of an illustrative embodiment of frangible connectors between the adjacent suture anchors of FIG. 8A in accordance with the present invention;

FIG. 8C shows an enlarged bottom perspective view of an illustrative embodiment of the frangible connectors of FIG. 8B in accordance with the present invention;

FIG. 8D shows a detail of an illustrative embodiment of breaking the frangible connection between the adjacent suture anchors of FIG. 8B in accordance with the present invention;

FIG. 9 shows an enlarged perspective view of an illustrative embodiment of a frangible connection between adjacent suture anchors with disengaging shoulders in accordance with an embodiment of the present invention;

FIG. 10A shows a perspective view of an illustrative embodiment of a needle delivery system for delivering and deploying a locked suture anchor in accordance with the present invention;

FIG. 10B shows a perspective view of an illustrative embodiment of a needle delivery system for delivering and deploying an unlocked suture anchor in accordance with the present invention;

FIG. 10C shows a perspective view of an illustrative embodiment of a needle delivery system piercing tissue for delivering and deploying a suture anchor in accordance with the present invention;

FIG. 10D shows a perspective view of an illustrative embodiment of a needle delivery system deploying a suture anchor in accordance with the present invention;

FIG. 10E shows a perspective view of an illustrative embodiment of a needle delivery system withdrawing from tissue after deploying a suture anchor in accordance with the present invention;

FIG. 10F shows a perspective view of an illustrative embodiment of a needle delivery system preparing a second suture anchor for delivery and deployment after deploying a first suture anchor in accordance with the present invention;

FIG. 10G shows a perspective view of an illustrative embodiment of a needle delivery system prepared for delivering and deploying a second suture anchor in accordance with the present invention;

FIG. 10H shows a perspective view of an illustrative embodiment of a needle delivery system locking suture in a second suture anchor in preparation for tensioning deployed suture in accordance with the present invention;

FIG. 10I shows a perspective view of an illustrative embodiment of a needle delivery system being prepared for tensioning deployed suture in accordance with the present invention;

FIG. 10J shows a perspective view of an illustrative embodiment of the needle delivery system of FIG. 10H with the locking tube advanced to lock suture within the second suture anchor in accordance with the present invention;

FIG. 10K shows a perspective view of an illustrative embodiment of the needle delivery system of FIG. 10J tensioning deployed suture in accordance with the present invention;

FIG. 10L shows a perspective view of an illustrative embodiment of a needle delivery system in preparation for cutting suture in accordance with the present invention;

FIG. 10M shows a perspective view of the illustrative embodiment of FIG. 10L with the locking tube advanced in preparation for cutting suture in accordance with the present invention;

FIG. 10N shows a perspective view of the illustrative embodiment of FIG. 10M with the locking tube rotated to cut suture in accordance with the present invention;

FIG. 10O shows a perspective view of an illustrative embodiment of a needle delivery system that includes a suture-cutting notch in accordance with the present invention;

FIG. 11A shows a perspective view of an illustrative embodiment of a suture lock in accordance with the present invention;

FIG. 11B shows a perspective view of an illustrative embodiment of a needle delivery system containing suture anchors and suture locks in accordance with the present invention;

FIGS. 12A-12E show an illustrative embodiment of a method for approximating tissue planes using a needle delivery system in accordance with the present invention;

FIG. 13 shows an illustrative embodiment of a method for repairing meniscus cartilage using a needle delivery system in accordance with the present invention;

FIG. 14 shows an illustrative embodiment of a handheld anchor delivery device for use with a needle delivery system in accordance with the present invention;

FIG. 15A shows an illustrative embodiment of a helical tissue control tool for use with a needle delivery system in accordance with the present invention;

FIG. 15B shows an illustrative embodiment of the helical tissue control tool of FIG. 15A positioned at a first tissue target site for use with a needle delivery system in accordance with the present invention;

FIG. 15C shows an illustrative embodiment of the helical tissue control tool of FIG. 15A positioned at a second tissue target site for use with a needle delivery system in accordance with the present invention;

FIG. 15D shows a schematic view of an illustrative embodiment of a suture-cutting device for the helical tissue control tool of FIG. 15A in accordance with the present invention; and

FIG. 16 provides a table of representative system parameters in accordance with various embodiments of the present invention.

### DETAILED DESCRIPTION OF THE INVENTION

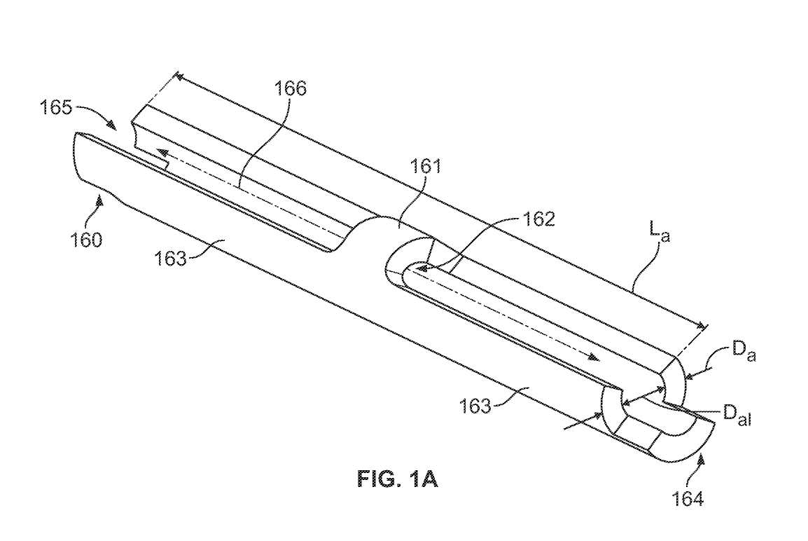

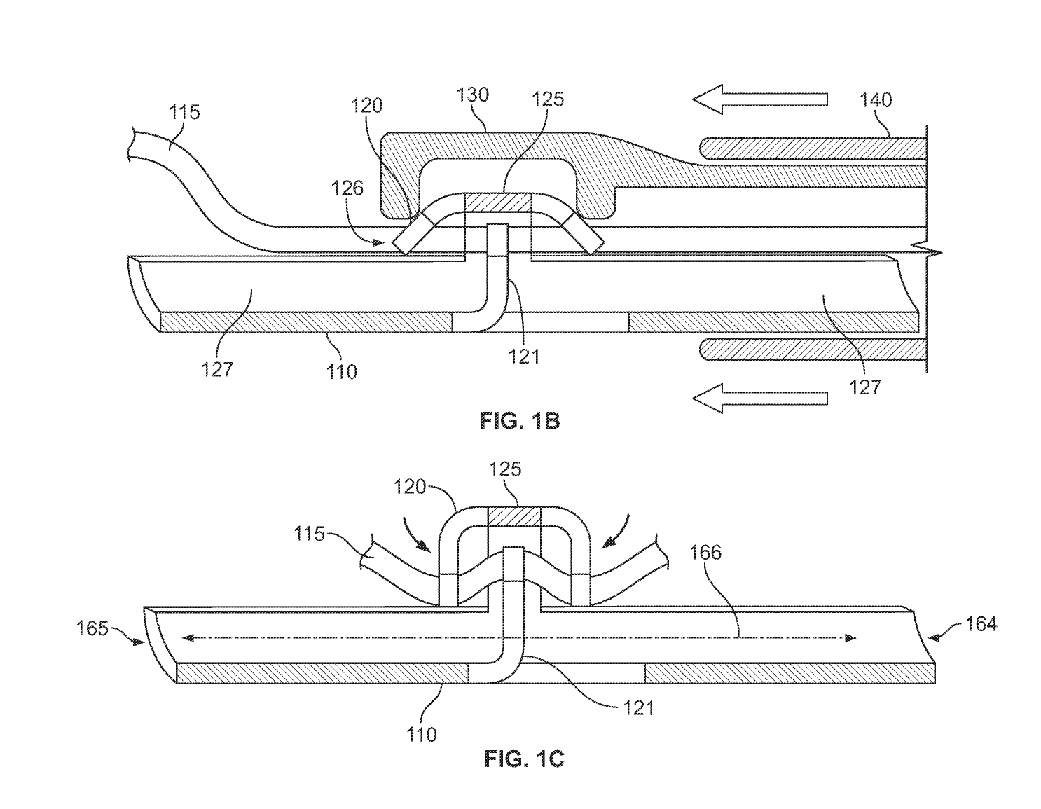

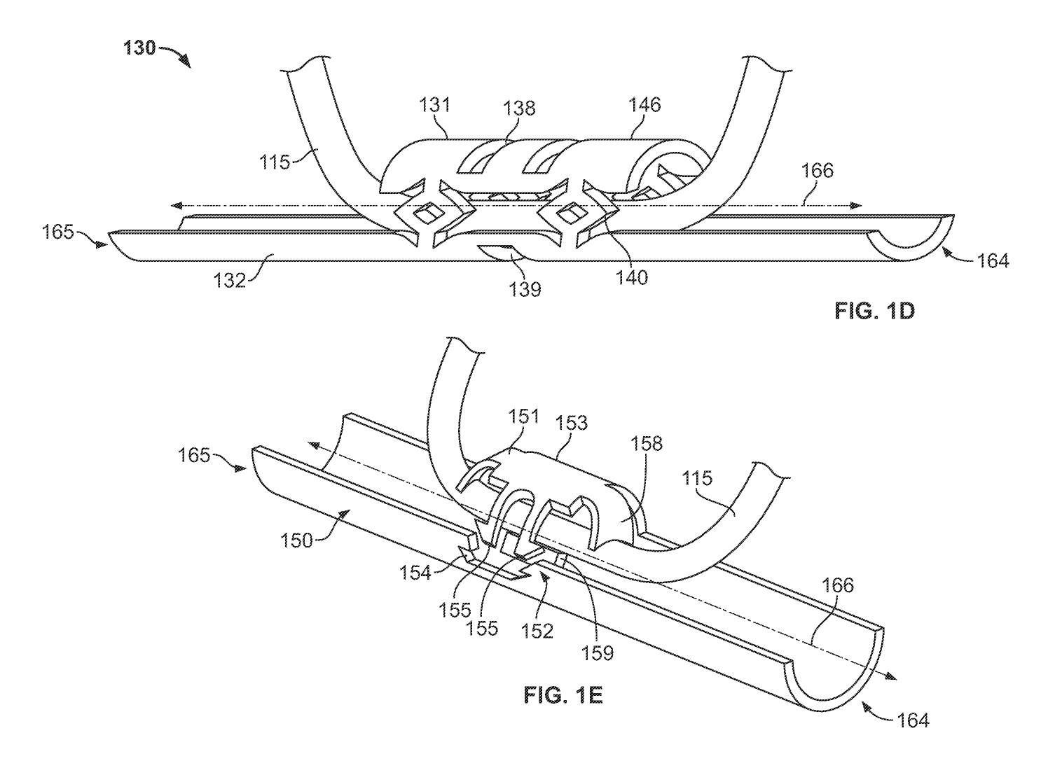

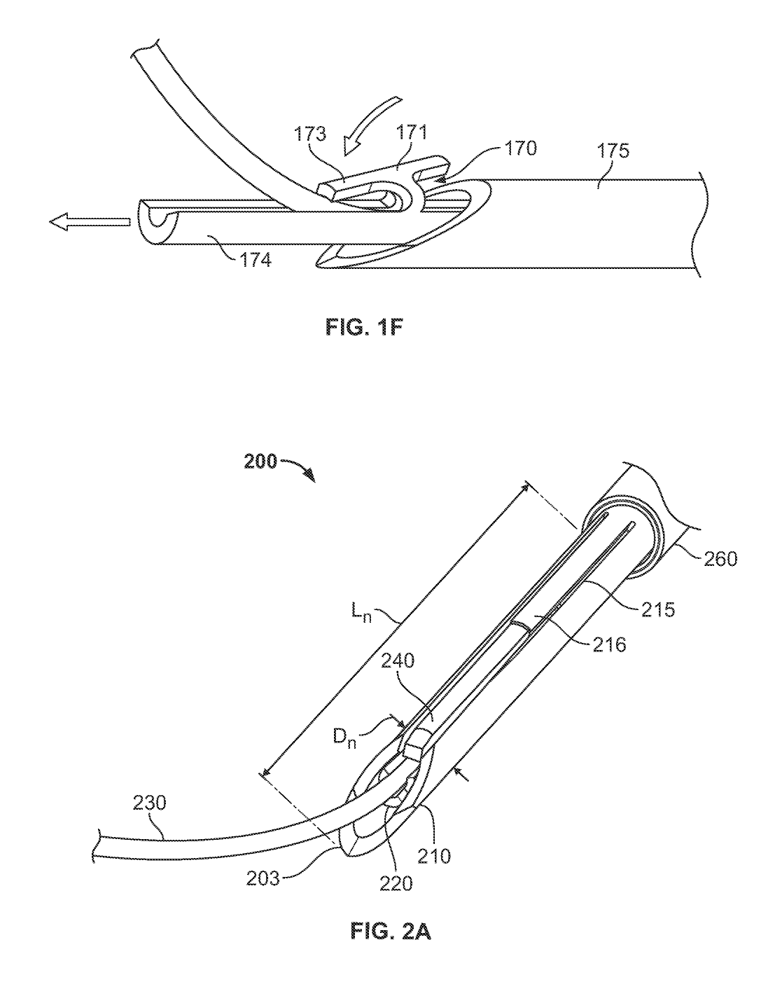

Referring to FIG. 1A, an illustrative embodiment of a selectively-lockable suture anchor 160 in accordance with the present invention is shown. In some implementations, the suture anchor 160 employs plastic or elastic deformation to lock suture within the lumen 162 of the suture anchor 160. Thus, in such implementations, a permanently-implantable, lockable suture anchor 160 may be made of, for example, metallic implant grade materials, e.g., stainless steel (304 grade steel), nickel-titanium alloy, cobalt chrome alloy (available from Elgiloy Specialty Metals), and the like, or implant grade polymers, e.g., PEEK, PEKK, polyamide, self-reinforcing polyphenylene, polyphenylsulfone, liquid crystal polymer, and the like. Alternately, in some implementations, suture anchors 160 may be designed to be bio-resorbable and may also be made of bio-absorbable metals, e.g., magnesium, iron, zinc, and so forth, or of bio-degradable polymers, e.g., poly-L-lactic acid (PLLA), polyglycolic acid (PGA), poly (D, L-lactide/glycolide) copolymer (PDLA), polycaprolactone (PCL), hyaluronic acid (HA), and the like.

A selectively-lockable suture anchor 160 may be laser cut from metallic or polymeric tubing, stamped from sheet metal, injection molded, laser-sintered from metallic or polymeric material, fused deposition modeled, 3D printed, or may be manufactured using any suitable method.

In some embodiments, the selectively-lockable suture anchor 160 is an elongate device having a proximal end 164, a distal end 165, a longitudinal axis (shown as dashed line 166) extending from the proximal end 164 to the distal end 165, a pair of lateral tissue supports 163, a centrally located suture loop or bridge 161, and a lumen 162 sized to slidably accept suture. The suture loop 161 and lumen 162 combine with the tissue supports 163 to provide a passage and mechanical clearance through which suture may freely and slidably travel bi-directionally. Advantageously, the suture loop 161 may be configured to slidably accept suture in an unlocked state but to be affixed to the suture in a locked state.

The lumen 162 is sized to slidably accept suture, but is only slightly larger in diameter than the diameter of the suture to be contained therein so that minor deformations of the suture loop 161 cause sufficient mechanical interference to lock the suture to the anchor 160. More particularly, the lumen diameter (Dₐ₁) is sized slightly larger than the diameter of the suture to be contained therein, leaving sufficient radial clearance within the lumen 162 for the suture to slide easily through the suture loop 161 and along the channel slots of the tissue supports 163. For example, for a non-resorbable 2-0 suture having a 0.012 inch diameter, suitable diameters of the anchor lumen 162 may range between about 0.013 and about 0.020 inches. Because the suture loop 161 is intended to be deformed or bent into the suture, in some variations the suture loop 161 may protrude radially above the slotted portions of the tissue supports 163 by an amount ranging between about 25 and about 50 percent of the lumen diameter (Dₐ₁).

Moreover, in some implementations, the slotted portions of the tissue supports 163 are approximately the width of the lumen diameter, while the open portions of the tissue supports 163 may make up at least 180 degrees of the circumference of the tissue supports 163 so that suture slips out of the slotted portions freely.

In some variations, the locked state may be achieved by crimping, compressing, deforming or otherwise displacing a portion of the suture loop 161 to eliminate mechanical clearance and to impinge upon suture within the lumen 162. Such deformation may be elastic or plastic or elasto-plastic. Alternately, locking may be achieved by forcing the suture into tight bends or a tortuous path within the suture loop 161 so as to increase friction between the suture and the anchor 160.

For example, in a first variation, referring to FIG. 1B, an elongate suture anchor 110 with suture 115 is shown. As previously described, suture 115 may be routed under the suture loop 125 and along the lumen 162. The suture anchor 110 may include tissue supports 127 that are separated by the suture loop 125, which may be selectively configured to engage a section of suture 115 to lock the anchor 110 to suture 115. In some implementations, the suture loop 125 may include one or more plastically deformable locking tabs 120. In some variations, the suture loop 125 may be axially aligned with at least one fixed tab 121 optionally having a V-shaped or U-shaped cutout or cradle to guide suture 115, forming a free-sliding aperture 126 along the longitudinal axis of the anchor 110 when in an unlocked state. In some instances, the locking tabs 120 may be positioned closely enough to one another on either side of the fixed tab 121 such that they may pinch the suture 115 against the fixed tab 121 when deflected into the locked position.

As shown in FIGS. 1B and 1C, locking tabs 120 may be selectively deflected into the path of the suture 115, e.g., by an anvil 130 of a delivery system displaced radially inward by a movable locking tube 140, such that the locking tabs 120 force suture 115 into a tortuous path about distal and proximal ends of the locking tabs 120 and the fixed tab 121 and/or mechanically engage the suture 115, e.g., against the distal and proximal ends of the locking tabs 120 and the fixed tab 121, sufficiently to prevent sliding and, thereby, locking the suture 115 relative to the anchor 110. As shown in FIG. 1C, a length of the locked suture portion extends in a direction of the anchor's longitudinal axis 166. The features and functionality of the delivery system are discussed in more detail below.

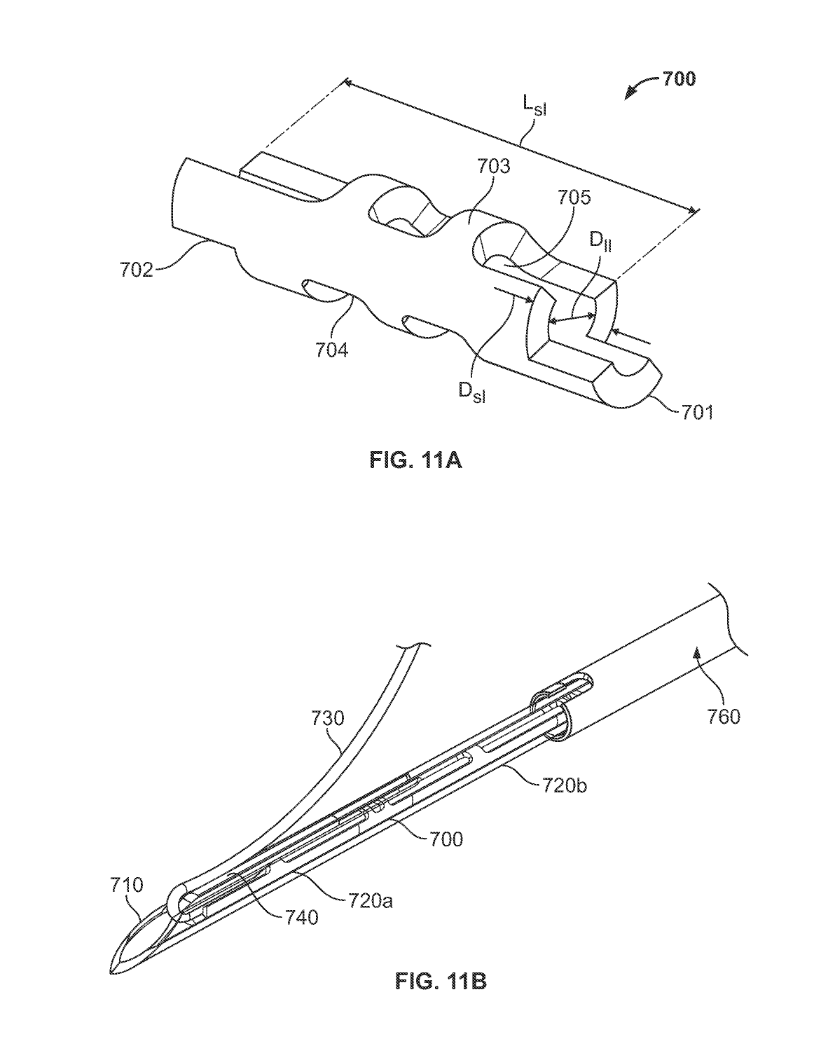

In some embodiments, a suture lock 700 (FIG. 11A), which is described in greater detail below, may be separate from the suture anchor 110 and may be used in conjunction with suture anchors 110 that are solely slidable with respect to the suture 115. In such embodiments, a selectively-engagable suture lock 700 may be included between each suture anchor 110 in a delivery system or at various intervals between a plurality of suture anchors 110 in the delivery system.

As shown in FIG. 1D, in another embodiment, a selectively-lockable suture anchor 130 includes a suture loop 146, one or more deformable connecting elements 140, and a fixed section 132 that provides tissue support. The suture loop 146 may include a movable section 131 having a plurality of suture engagement features 138, e.g., suture engagement slots, suture engagement teeth, or other friction-enhancing features. The fixed section 132 may also include such suture engagement features 139. The movable 131 and fixed sections 132 are connected by the one or more plastically deformable connecting elements 140, which are structured and arranged so that, once a force has been applied to the movable section 131, e.g., by the anvil 130, the suture engagement features 138 and 139 are brought closer together to engage suture 115. The suture engagement features 138, 139 may be configured as a contiguous circumferential structure including deformable elements or may be configured as a discontinuous gapped structure capable of permanent deflection through plastic deformation. Again, as shown in FIG. 1D, a length of the locked suture portion extends in a direction of the anchor's longitudinal axis 166.

In yet another embodiment, as shown in FIG. 1E, a selectively-lockable suture anchor 150 includes a suture loop 153 having a plurality of elastically-deflectable locking features, e.g., prongs 155, on a movable (e.g., hinged) section 151, and a fixed section 152 having engaging locking features, e.g., teeth 154. In a locked position, the prongs 155 of the suture loop 153 and the teeth 154 of the fixed section 152 may be in engaging contact to lock the movable section 151 to the fixed section 152. When so engaged, locking tabs 158 disposed at distal and proximal ends of the suture loop 153 and one or more fixed tabs 159 disposed therebetween on the fixed section 152 may lock a portion of the suture 115 so that a length of the locked suture portion extends in a direction of the anchor's longitudinal axis 166. As in previously described embodiments, the selectively-lockable suture anchor 150 may be configured with contiguous circumferential structural elements capable of accommodating displacement through deformation and/or the selectively-lockable suture anchor 150 may be configured as a discontinuous gapped structure held together by slidable interlocking elements.

In still another embodiment, as shown in FIG. 1F, the suture anchor 174 may initially be constrained in an unlocked state within the lumen of a delivery needle 175. The locked state of the suture anchor 174 may be achieved by releasing an elastically-deformed portion 170 of a suture loop 171 such that a portion 173 of the elastically-deformed portion 170 impinges radially inwards upon the suture. The mechanical friction created by elastic constraint of the suture loop 171 may also prevent the anchor 174 from prematurely slipping out of the delivery needle 175.

The selectively-lockable suture anchor 150 may be cut from tubing by laser or equivalent processes with the locking tabs 158, 159 subsequently formed into the desired position. Alternatively, the suture anchor 150 may be stamped from sheet material and formed via progressive die stamping into the final shape.

Advantageously, the suture locking mechanism or structure may be included in a suture anchor, such that the fixed and/or the movable section of the suture lock includes projections that extend axially away from the lock in both directions, thus forming a T-shaped suture anchor.

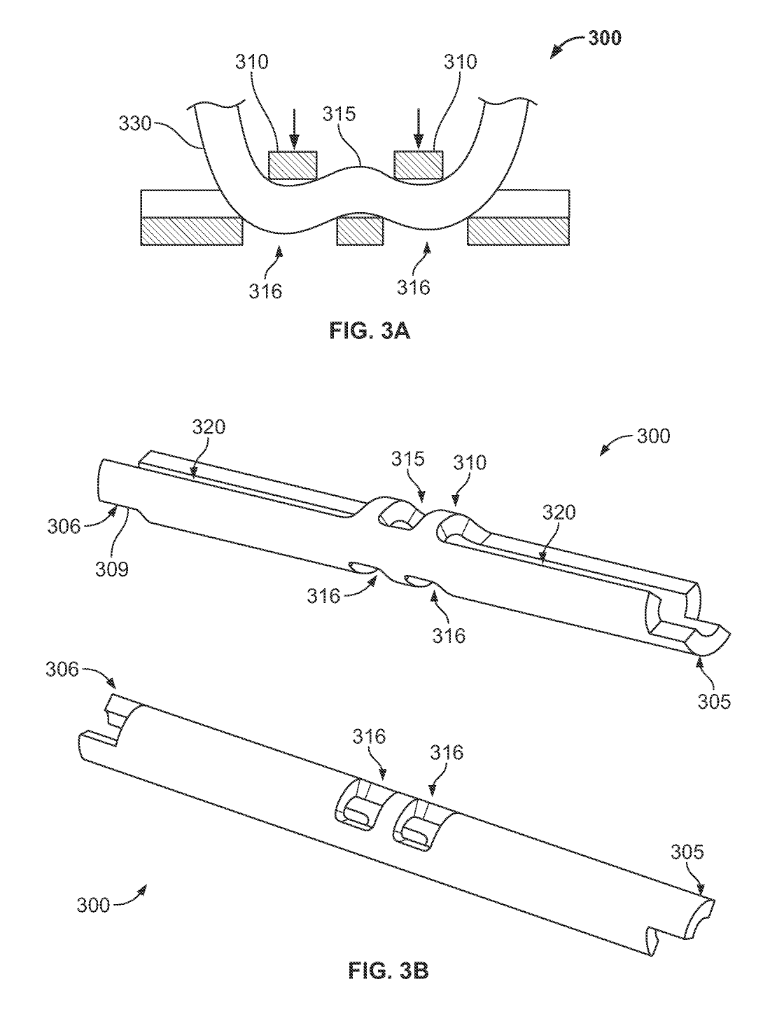

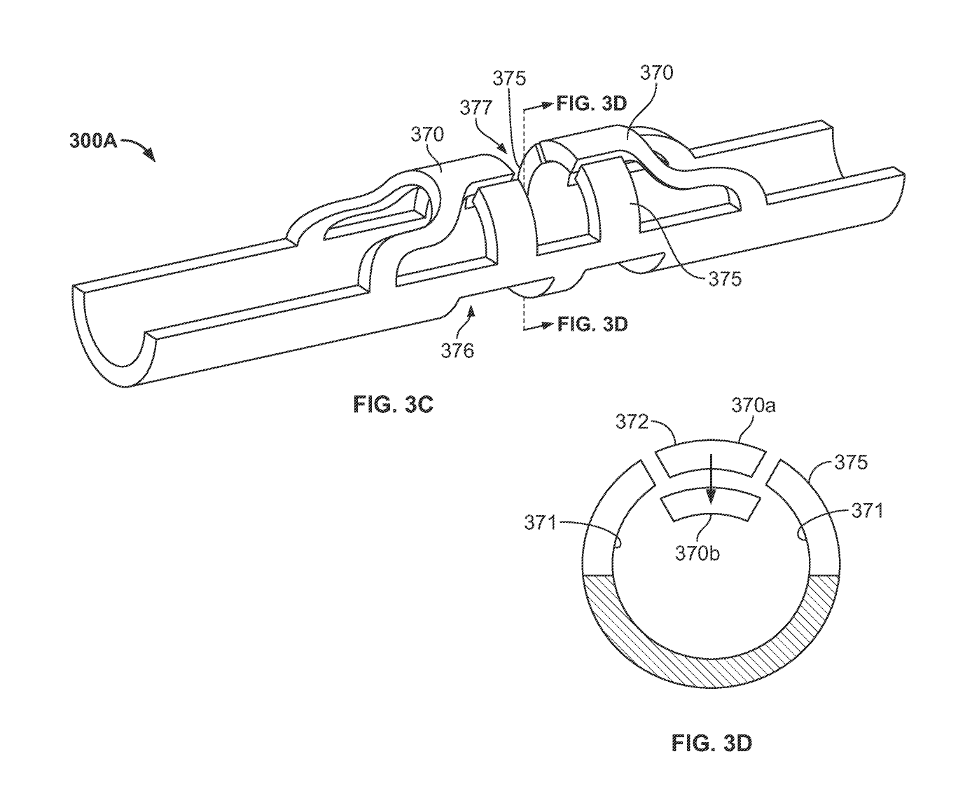

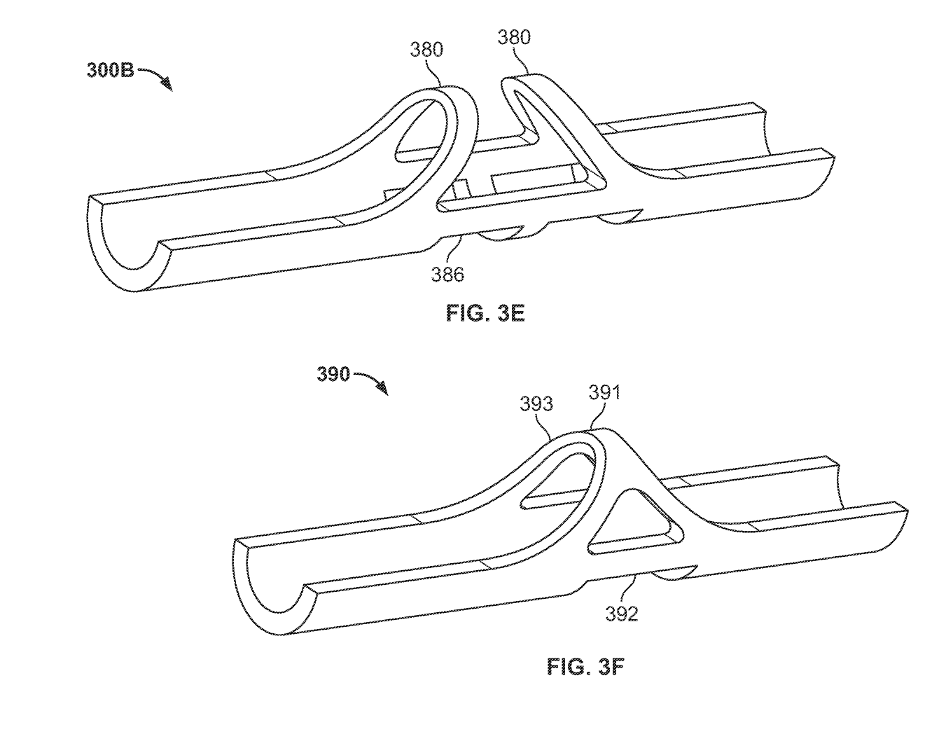

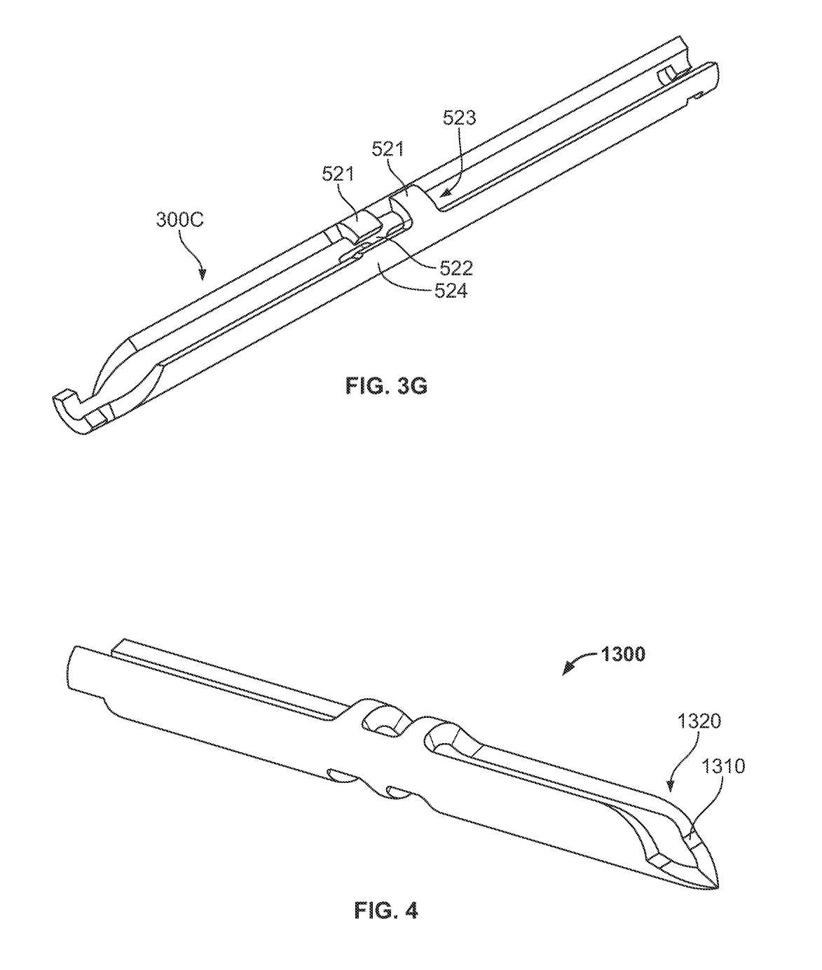

An illustrative embodiment of a double-wiggle-type suture anchor is shown in FIGS. 3A and 3B. In FIG. 3A, suture 330, which, initially, slidably extends through the lumen of a suture anchor 300, may be reliably locked to the anchor 300 by deflection of at least two bridge elements 310 into the anchor lumen. Inward deflection of the bridge elements 310 displaces a portion of suture 330 into corresponding gaps formed as lower suture pocket(s) 316. A gap between adjacent bridge elements 310 may form an upper suture pocket 315. Multiple bridge elements 310 deflected toward lower suture pockets 316 force the suture 330 into a zigzag or undulating pattern (e.g., W-shaped) that increases friction between the suture 330 and the anchor 300 sufficiently to functionally lock suture 330 to the anchor 300. This configuration may be advantageous in enabling larger, less precise radial displacements of a locking suture loop or bridge 310 with reduced risk of severing the suture 330. Referring to FIG. 3B, longitudinal openings on either side of the bridge elements 310 provide suture slots 320, which may be sized to allow suture 330 to slip laterally, e.g., out of the anchor lumen, and around the bridge element 310 such that the installed anchor and suture adopt a T-shape in tissue when the suture is tensioned.

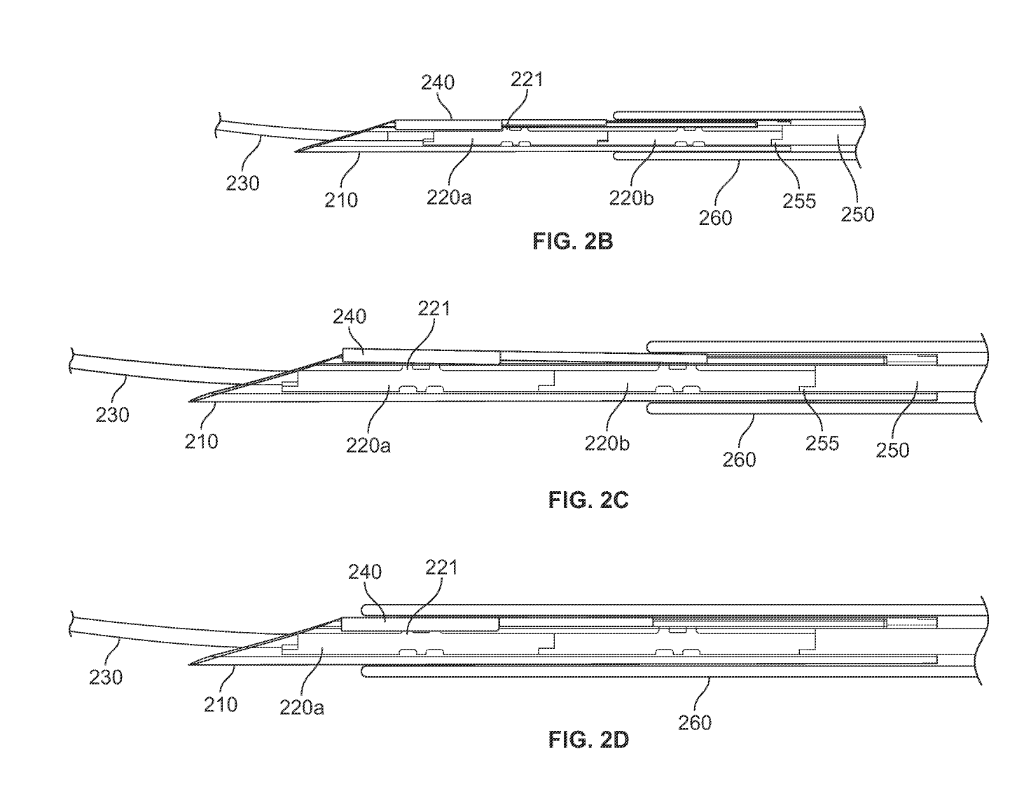

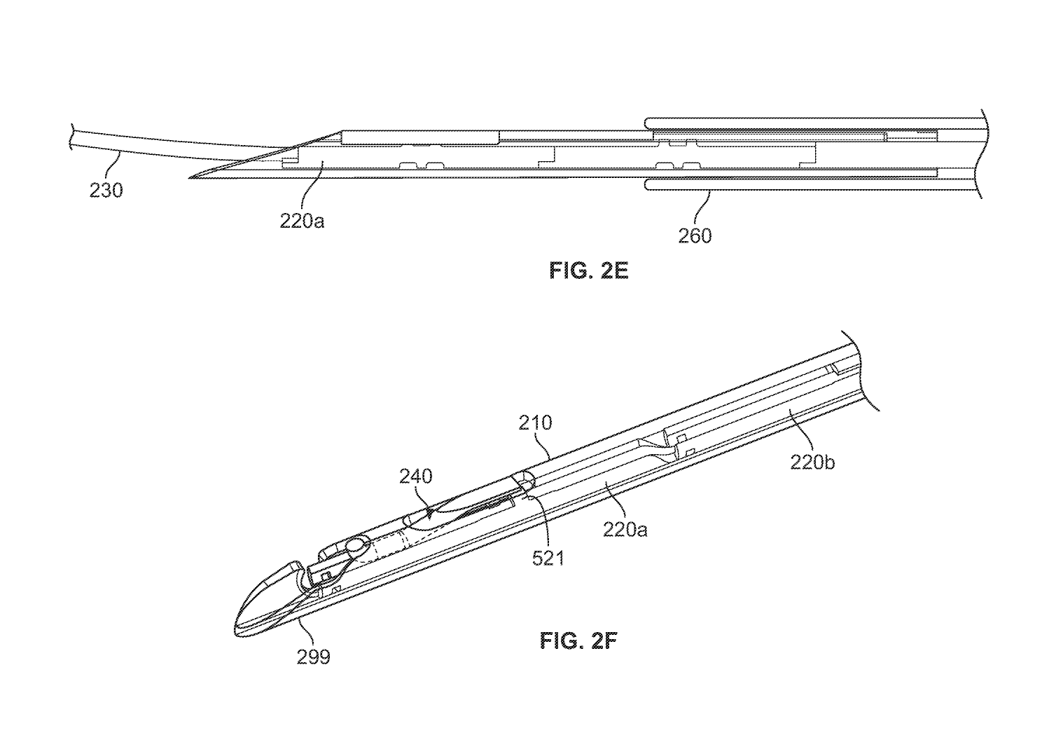

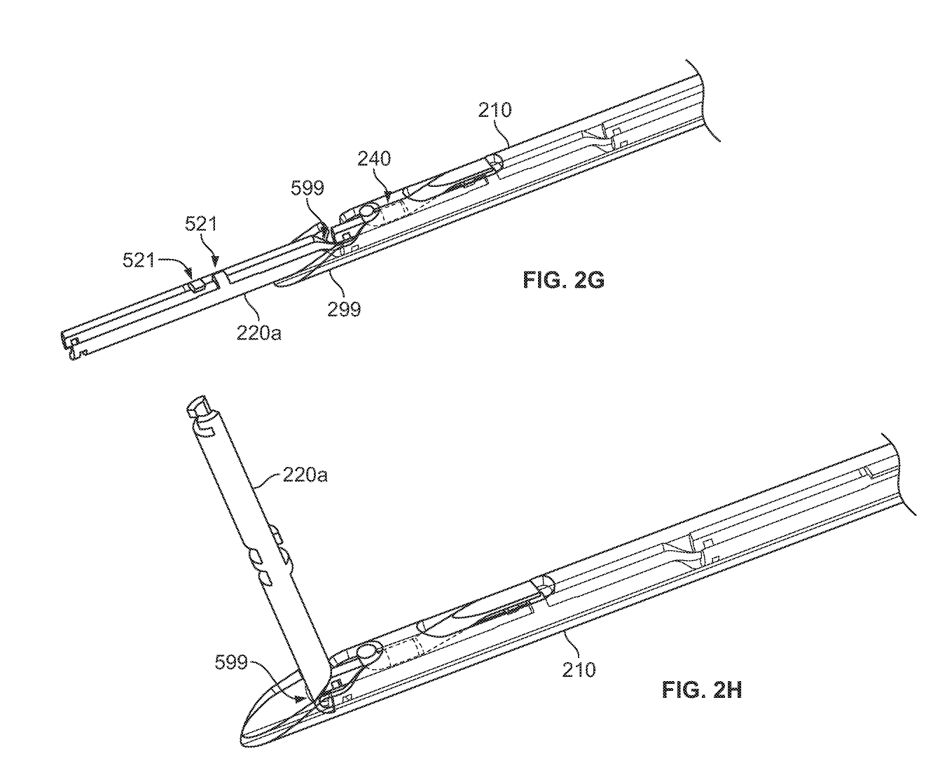

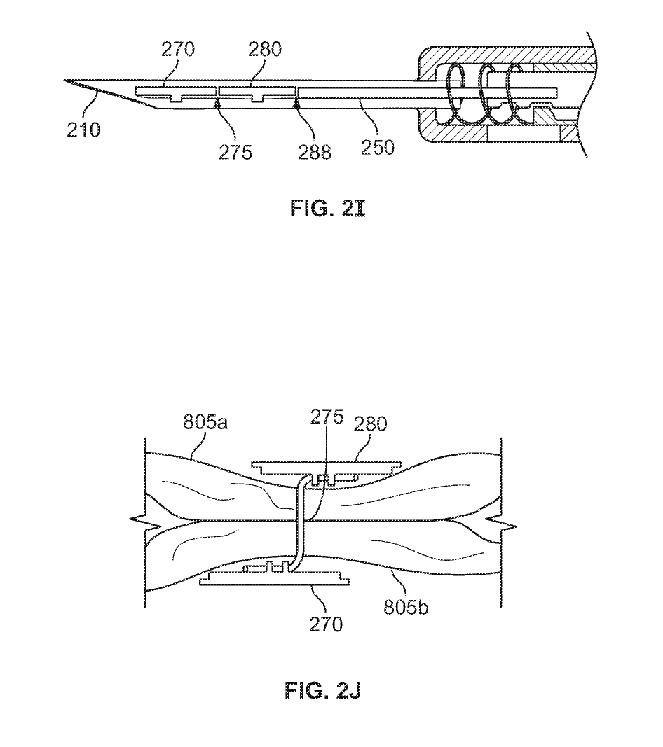

As shown in FIG. 3B, the suture anchor 300 may also include anti-rotation features 305 and 306 that may be configured to substantially lock anchors together circumferentially so as to prevent relative rotation. In some variations, a female anti-rotation key 306 may include an undercut feature 309 that may be dimensioned to receive the male anti-rotation key 305 of an adjacent suture anchor 300. Thus, undercut, interlocking anti-rotation features 305, 306 may mechanically lock abutting anchors 220a, 220b to each other along the axial or longitudinal dimension as shown in FIG. 2B while they are constrained within a delivery needle 210. Once a distal anchor 220a has substantially exited the delivery needle 210 it is free to separate from the more proximal anchor 220b and deploy from the delivery system 200. This configuration may be advantageous in preventing anchors 220a, 220b from slipping away from each other and losing rotational alignment within the delivery needle 210. Interlocking anti-rotation features 305, 306 may also help prevent the distal anchor 220a from unintentionally slipping out of the distal tip of the delivery needle 210.

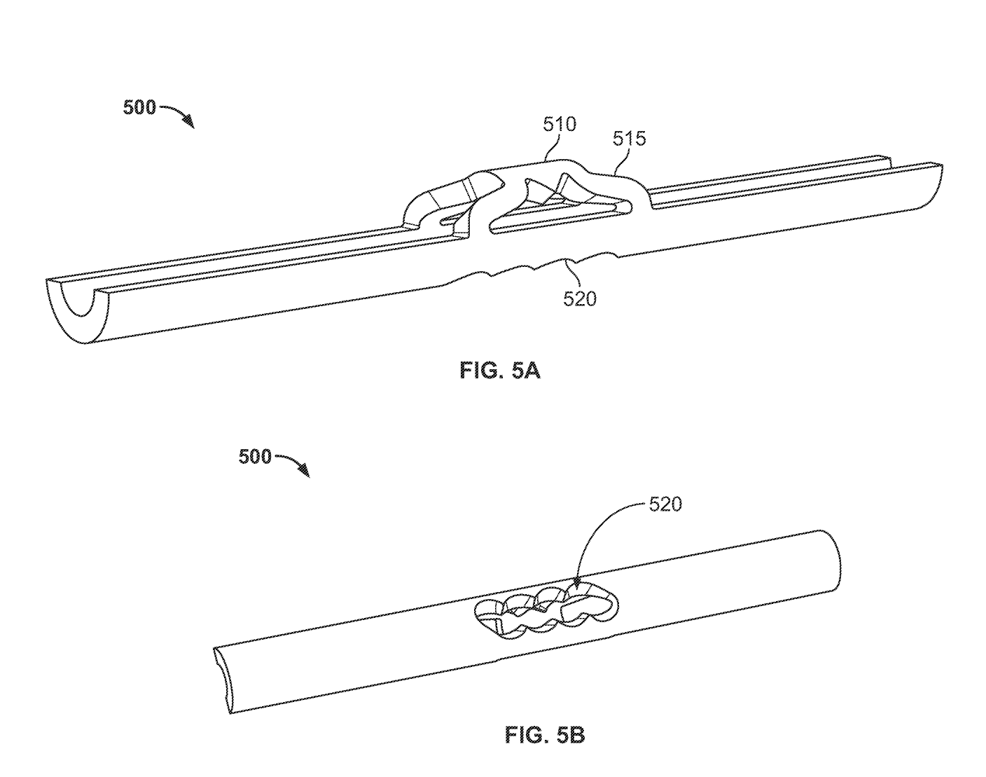

An illustrative embodiment of a suture anchor 300A including a mechanical suture locking mechanism employing sliding surfaces and elastic deformation to hold a bridge 370 in either a radially outward or radially inward position is shown in FIGS. 3C and 3D. Mechanical suture locking may be advantageous in producing a firm (and optionally reversible) suture lock with relatively lower locking forces compared to crimping suture locking or other plastic deformation-based suture locking Such a mechanical suture locking mechanism may include a deflectable bridge 370 having a wedge-shaped cross section that is dimensioned such that the radially inner edge 371 of the flexure opening is smaller than the radially outer edge 372 of the bridge 370. As a result, when forceably moved radially inwards against similarly wedge-shaped surfaces of locking flexure 375, the bridge 370a may elastically spread the ends of the locking flexure 375 and become captured by the flexure 375 once the ends spring back around the bridge 370a. In some variations, once locked in the radially inward position, a gap between two bridge elements 370 may form an upper suture pocket 377 and the bridge 370b may maintain deflection of a portion of suture into a lower suture pocket 376 to increase friction and lock suture to the anchor 300A. In some implementations, the lower pocket 376 may also include relatively sharpened features to increase longitudinal friction, e.g., a toothed slot 520 as shown in FIG. 5B, which will be discussed in greater detail below.

In one variation, as shown in FIG. 3E, bridge 380 elements may be angled with respect to the longitudinal axis of a suture anchor 300B. This configuration may be advantageous in reducing the amount of radial mechanical force required to deform the bridge 380 radially inwards, such that it may displace a section of suture into a lower suture pocket(s) 386. In yet another variation, as shown in FIG. 3F, a lockable suture anchor 390 may include a single bridge 391 and a single suture pocket 392. For example, the bridge 391 may be supported by plastically deformable angled struts 393 configured to allow the bridge 391 to move radially inward such that it may push a portion of suture into the lower suture pocket 392.

In yet another variation, a sharp-tip suture anchor 1300 may be employed. For example, as shown in FIG. 4, an illustrative embodiment of a suture anchor 1300 may include a sharpened distal tip 1310. Advantageously, with this configuration, suture anchors 1300 may first assist tissue penetration in order to place the suture anchor 1300 into or through the desired tissue and then function to anchor suture by rotating into a broad surface area T position with respect to the entry hole. The suture anchor 1300 may include a relatively blunt, e.g., rounded or radiused, shoulder 1320 located proximally to the sharp distal tip 1310. This shoulder 1320 may be used to transmit axial force between a series of anchors within a delivery needle while protecting the sharp distal tip 1310. The rounded shoulder 1320 minimizes resistance as the portion of the anchor proximal to the sharp tip 1310 passes into the puncture site. Intermeshing anchor tips 1310 with trailing ends of adjacent anchors may also serve to limit relative rotation between anchors. The sharp tip configuration of anchor 1300 can be advantageous in minimizing the overall size of the tissue puncture by enabling the suture anchor 1300, rather than the larger delivery needle, to form its own puncture and may find particular utility in thin or delicate tissues.

In still another variation, as shown in FIGS. 5A and 5B, a suture anchor 500 may include a bridge 510 supported by plastically deformable struts 515 in combination with a toothed slot 520. In use, the bridge 510 may be displaced into the suture anchor's central lumen by plastically deforming struts 515, forcing some portion of suture into the toothed slot 520, which, in some implementations, may be sized to be narrower than the suture diameter. The bridge 510 may optionally include a suture-facing toothed portion, as depicted. Deformation of a section of relatively softer suture into the relatively harder toothed slot 520 further ensures locking suture to the anchor 500.

In still another variation, as shown in FIG. 3G, a suture anchor 300C may include one or more cantilevered tabs 521 configured, when deformed, e.g., by an anvil, to lock a portion of suture within a central lumen 523. Although FIG. 3G shows two cantilevered tabs 521, this is done for the purpose of illustration and not limitation. Indeed, those of ordinary skill in the art will appreciate that the number of cantilevered tabs 521 may be more or less than two. The gap 522 separating the distal (i.e., cantilevered) end of each cantilevered tab 521 from the side wall 524 of the suture anchor 300C may be sized to prevent suture from slipping out of the lumen 523 while the cantilevered tab 521 is undeformed (i.e., prior to the tab 521 being bent radially inward, e.g., by an anvil, to compress and lock a portion of suture). In some variations, gap 522 is further sized such that bent or deformed cantilevered tabs 521 do not mechanically interfere with the side wall 524 of the anchor 300C after the tabs 521 have been bent or deformed.

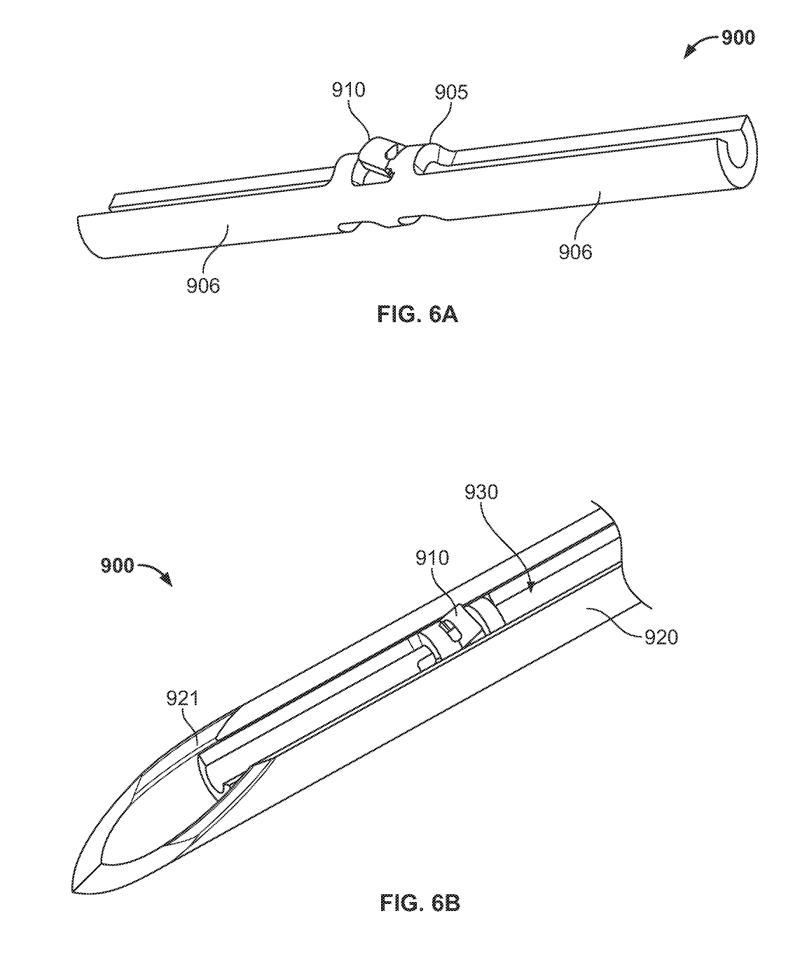

In some applications, for example, in which there are multiple anchors disposed within a delivery needle, it may be advantageous to maintain rotational alignment of suture anchors within the delivery system. Accordingly, one or more structural features may be integrated into the suture anchor to aid in aligning the anchor with other anchors along a longitudinal length of the delivery system. In one embodiment, as shown in FIGS. 6A and 6B, one or more suture anchors 900 may include a protruding tab 910 that is configured to engage an alignment slot 930 formed in the wall of a delivery needle 920. The protruding tab 910 may be located anywhere on the anchor 900, including centrally at the suture loop 905 or anywhere along one or both of the tissue supports 906. While the alignment slot 930 may be located anywhere on the circumference of the delivery needle 920, in some embodiments it is located within the non-sharpened heel 921 of the distal tip of the delivery needle 920. In some embodiments, the alignment slot 930 may terminate proximal to the needle bevel such that protruding tab 910 must be deflected radially inwards to exit the delivery needle 920.

In another embodiment, the slot 930 edges may be inverted or everted to engage a portion of each anchor and maintain rotational alignment. In various other embodiments, the delivery needle and anchors may have an asymmetrical cross-section that may include a non-cylindrical portion, e.g., a D-shape or other flat surface, that prevents rotation. Alternatively, the delivery needle and anchor may have a symmetrical faceted cross-section, e.g., a square, hexagon, or the like, to maintain anchor alignment. Additionally, each anchor may have radial ridges at the proximal end that directly engage and mate with corresponding radial ridges in the distal end of the next proximal anchor.

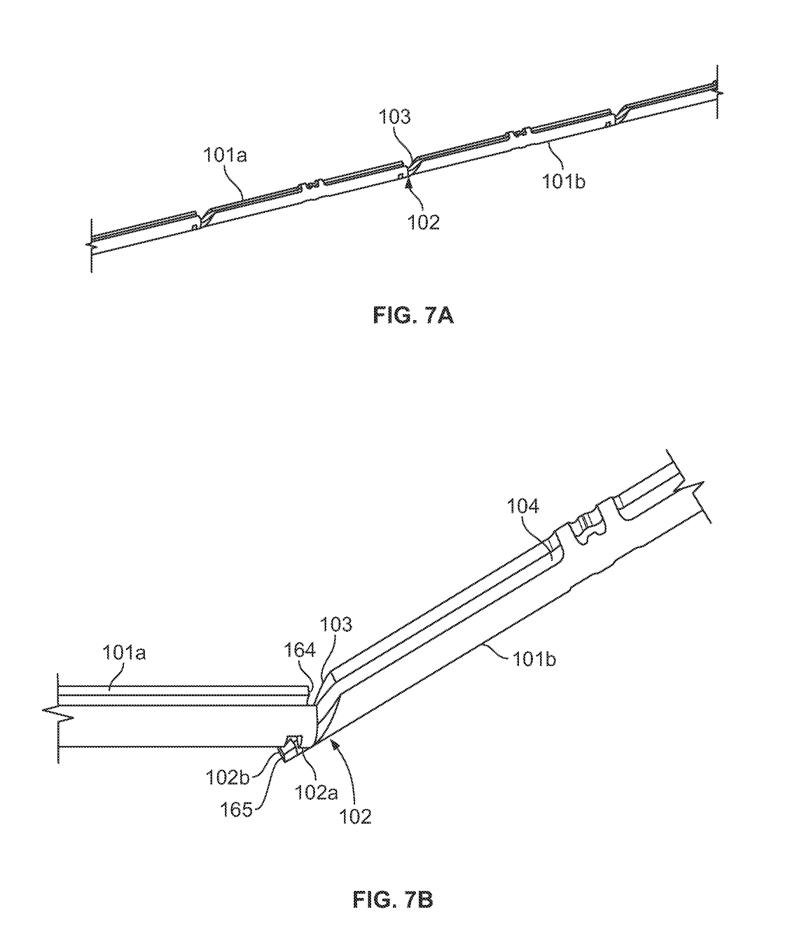

In another variation, referring to FIGS. 7A and 7B, selectively-lockable suture anchors 101, arranged end-to-end to facilitate loading into a delivery needle, may be interconnected, e.g., by interlocking links 102 disposed at distal 165 and proximal ends 164 of each suture anchor 101. In addition, at its distal end 165, each anchor 101 may include an angled face 103 (i.e., a beveled distal tip), allowing sufficient clearance for rotation to occur between adjacent anchors 101a, 101b (e.g., in a single plane). The link features 102a, 102b are located radially from each anchor's longitudinal axis. Tension applied to suture extending through the anchor's central lumen 104 creates a moment, tending to pivot the anchor 101a (e.g., in a single plane) at the point of juncture of interlocking features 102 facilitating disengagement.

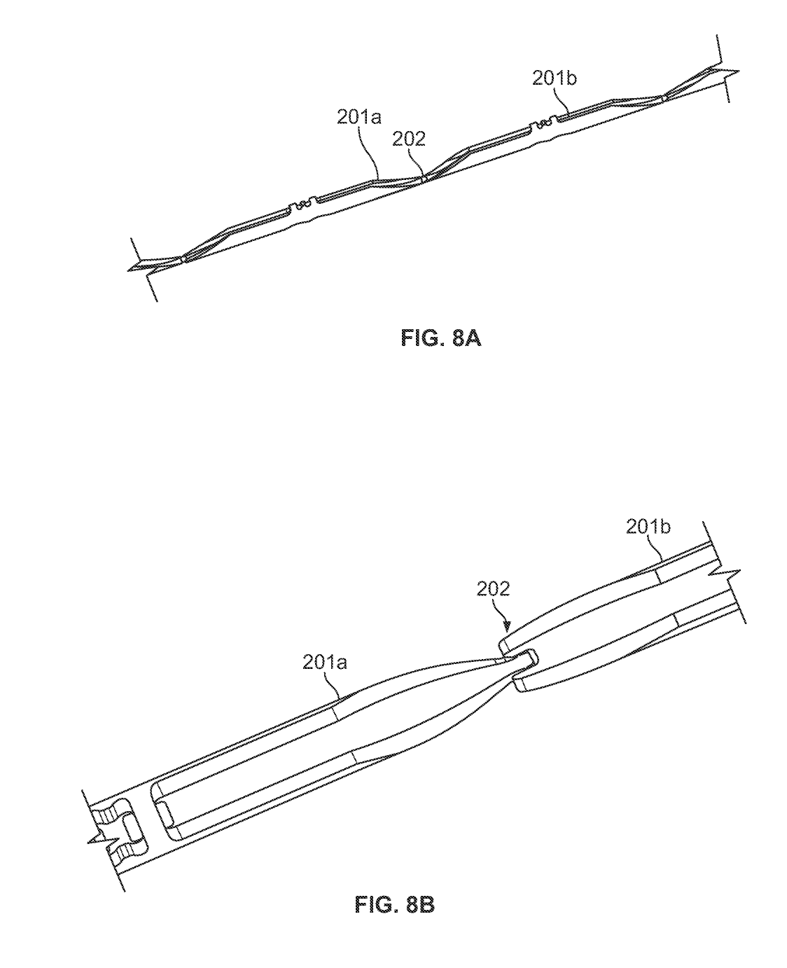

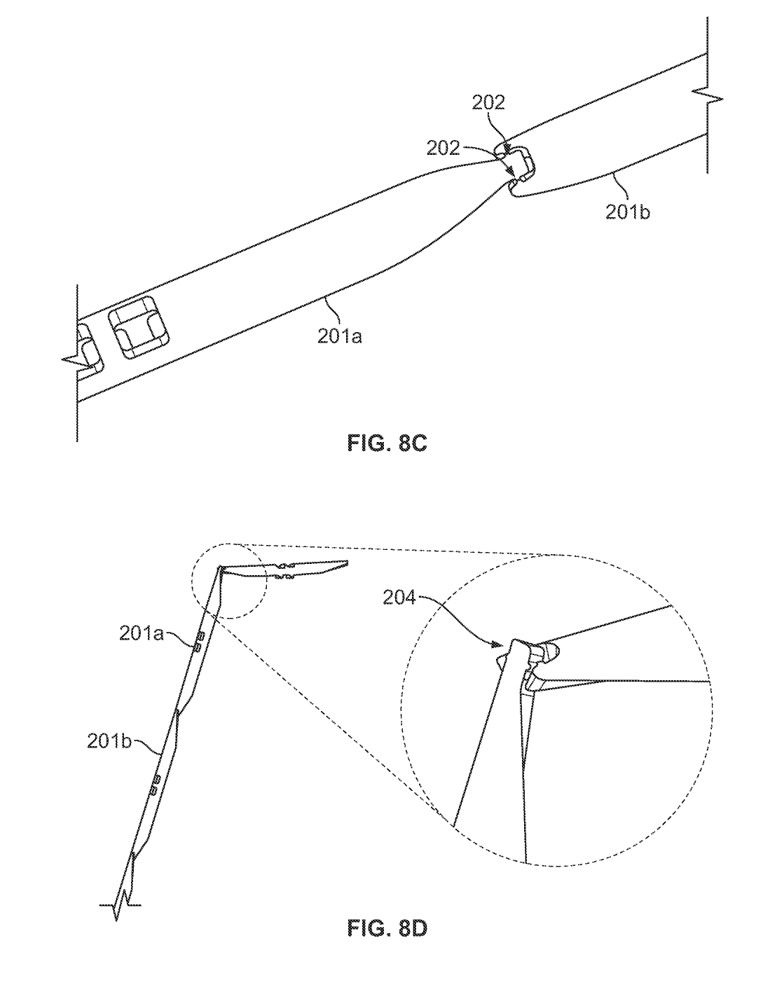

In another variation, as shown in FIGS. 8A-8D, a series of suture anchors 201 may be formed from a single piece of material, e.g., stainless steel hypo tubing, bio-erodible materials (such as magnesium), bio-absorbable materials (such as hyaluronic acid), and the like. The anchors 201 may include one or more frangible connectors 202 configured to also act as rotational hinges. In some implementations, the connectors 202 may be configured such that hinging rotation may mechanically strain the connectors 202 to the point of fracture, thus severing the connection between adjacent anchors 201a, 201b.

In some embodiments, the connectors 202 may be oriented relatively circumferentially to form a hinge between anchors 201a, 201b such that the connectors twist upon relative rotation of adjacent anchors 201a, 201b. The connectors 202 may be relatively short and narrow so that mechanical strain may be concentrated on the connectors themselves so as to promote work hardening and, as shown in FIG. 8D, fracturing 204 upon rotation. Furthermore, in some embodiments, the connectors 202 may be located substantially laterally on the same side of the central axis of a line or queue of suture anchors 201, such that tension on suture within the central lumen may produce a torqueing moment tending to rotate adjacent anchors 201a, 201b around the connectors 202.

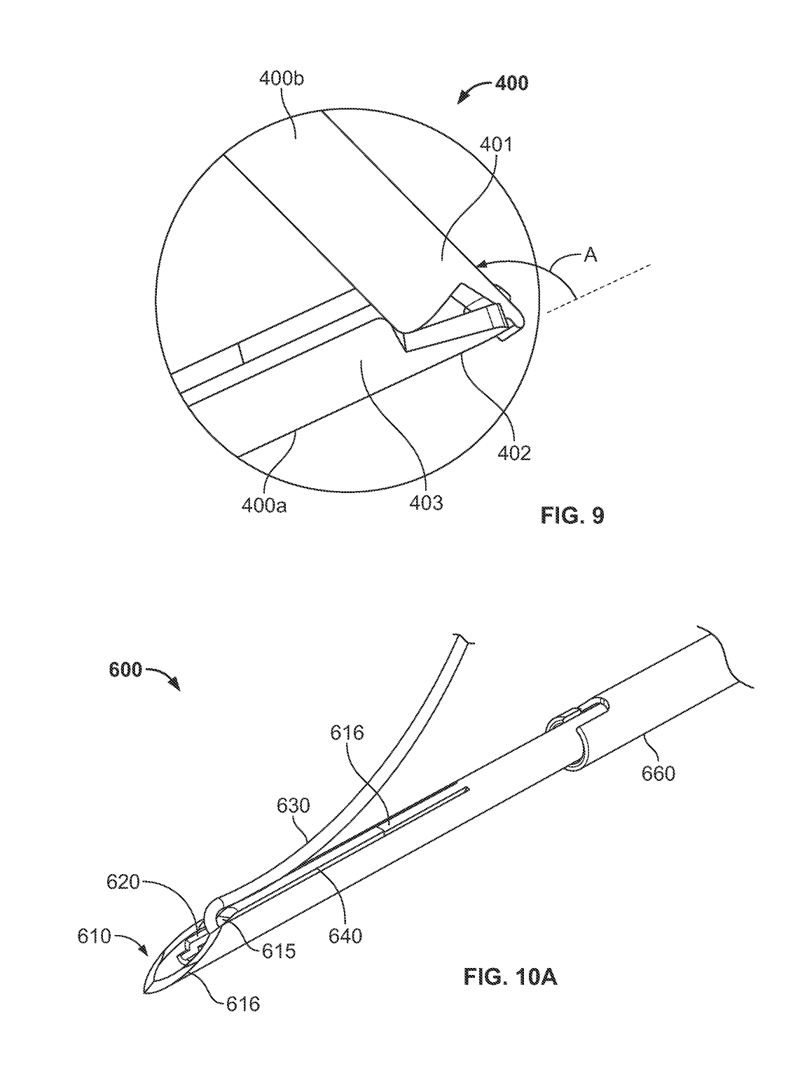

In some implementations, referring to FIG. 9, frangible suture anchors 400 may be structured and arranged to include disengagement shoulders 401 and 402 configured to mechanically abut at a contact point 403, e.g., after a certain angular displacement has occurred and the connectors have fully fractured but not yet separated. The shoulders 401, 402 may be configured such that the contact point 403 forms a fulcrum of a lever that tends to shift the fractured remnants of the connectors away from each other as rotation continues, thus freeing the newly severed anchors 400a, 400b from each other. For example, contact may occur at a relative rotation angle A in a range of between about 90 degrees and about 160 degrees, e.g., at about 110 degrees, between anchors.

Having described a number of embodiments of selectively lockable suture anchors with a variety of locking mechanisms, systems for deploying and delivering suture anchors and/or other medical devices will now be described in detail. Referring back to FIG. 2A, an illustrative embodiment of an in-plane, double-wiggle-type anchor deployment and delivery system 200 is shown. In some implementations, the system 200 may include a delivery needle 210 that may be enclosed within and coaxial with a locking tube 260. The delivery needle 210 may be hollow with a cylindrical or substantially cylindrical shape to form a lumen for receiving the anchors and suture. Although the shape of the delivery needle 210 has been described as cylindrical or substantially cylindrical, that is for illustrative purposes only. Those of ordinary skill in the art will appreciate that the delivery needle 210 and its lumen may have any shape, as long as the shape enables the delivery needle 210 to fit within the lumen of the locking tube 260 and to serve its purpose during a surgical procedure.

At a distal end 203, the delivery needle 210 may have a sharpened distal tip, e.g., for piercing the epidermis, tissues, etc. of a patient, and an opening through which a suture anchor 220, suture 230, a medical device, and so forth, may exit the delivery needle 210. The lumen formed by the delivery needle 210 provides space through which suture anchors 220, suture 230, medical devices, and so forth may slidably pass. Suture anchors 220 may be sized with sufficient radial clearance to facilitate a close sliding fit within the lumen of the delivery needle 210 as well as to allow suture 230 to slide within and through each suture anchor 220.

In some applications, the delivery needle 210 may include at least two slots 215, extending longitudinally in the cylindrical wall to the distal needle tip of the delivery needle 201. The slots 215 may be configured to provide a cantilever 216 at a distal end of which is an anvil 240 that may be thicker (in a radial direction) than the cylindrical wall of the delivery needle 201 and optionally contoured, depending on the configuration of the anchor locking feature. The cantilever 216 enables the anvil 240 to displace radially within the delivery needle 210, when a radial force is applied to the anvil 240. As a result, the cantilever 216 may be configured to deform elastically as the anvil 240 is radially displaced either outwards or inwards. In addition to, for example, crimping or pinching the locking tab 120 of the suture lock (or the other displaceable sections of the various locking mechanisms described above), the anvil 240 may also be configured as a stop, to prevent the next suture anchor 220 from slipping out of the delivery needle 210, that protrudes into the central lumen of the delivery needle 210 and into the path of a portion of the distal anchor 220a.

More particularly, as shown in FIG. 2B, when the anvil 240 and cantilever 216 are in their normal operating position, the thicker radial dimension of the anvil 240 is adapted to interfere with one or more anchor bridges 221 (or other displaceable sections of the various locking mechanisms described above) on the distal suture anchor 220a. During deployment, the anvil 240 may be displaced radially outwards, e.g., by advancing the distal suture anchor 220a distally, such that the anchor bridge(s) 221 pushes against and overrides the anvil 240, displacing the anvil 240 radially outwards. During a suture locking sequence, the anvil 240 may be displaced radially inwards, e.g., by advancing the close fitting locking tube 260 over the anvil 240 and the delivery needle 210. Anchor locking tube 260 may be proximally withdrawn along the delivery needle 210 such that it exposes the needle tip for tissue puncture or be advanced distally toward the needle tip to press the anvil 240 radially inwards into an anchor 220a to lock the suture anchor 220a to suture 230.

The system 200 may also include a pushrod 250 to advance one or more suture anchors 220 axially or longitudinally through the delivery needle 210, as well as to prevent anchors 220 from sliding back out of the delivery needle 210. As shown in FIG. 2C, the pushrod 250 may advance suture anchor 220a distally, such that the distal anchor bridge 221 slides along and overrides the inward-facing surface of the anvil 240. When sufficient force is applied to the anchor 220a by the pushrod 250, the bridge(s) 221 displaces the anvil 240 radially outward, resulting in a portion of anvil 240 radially protruding from the outer surface of the delivery needle 210. Advantageously, in some variations, the pushrod 250 may include an anti-rotation key 255 adapted to mate with a corresponding anti-rotation feature integrated into a proximal suture anchor 220b, to prevent relative rotation of the anchors 220a, 220b within the lumen of the delivery needle 210.

The pushrod 250 may be grooved or hollow, so that suture 230 may extend slidably through the pushrod 250, as well as through the suture anchors 220 and the delivery needle 210. In one embodiment, as illustrated in FIG. 2C, the delivery needle 210, each anchor 220, and the suture 230 are aligned substantially coaxially. In some embodiments, one end of the suture 230 may extend out of the proximal end of the delivery needle 210 and locking tube 260 such that it may be manually accessible to surgeons or other medical personnel, e.g., so that a suture stitch may be tightened. In other embodiments, a proximal section of suture 230 may be gripped mechanically and stitches may be tightened via a pulling mechanism included in a surgical tool, such as the tool described in greater detail below.

In certain circumstances, e.g., when preparing an anchor for deployment through a tissue layer, it may be preferable to advance the distal suture anchor 220a a sufficient distance to deflect anvil 240 radially, while retaining the suture anchor 220a entirely within the delivery needle 210, as shown in FIG. 2C. As shown in FIG. 2D, once the distal suture anchor 220a has been pushed proximate the opening of the delivery needle 210, the locking tube 260 may be advanced distally such that, as the locking tube 260 nears the distal end of the delivery needle 210, the inner surface of the locking tube 260 forces the protruding anvil 240 radially inwards, thereby deforming one or more anchor bridges 221 and advantageously locking the distal anchor 220a to suture 230. As shown in FIG. 2E, after locking suture 230 in the distal suture anchor 220a, the locking tube 260 may be withdrawn, e.g., by drawing it back proximally. Naturally, other anchor locking mechanisms can be similarly actuated by cooperation of the pushrod 250, anvil 240, and locking tube 260.

Referring to FIGS. 2F through 2H, an alternative embodiment of a needle delivery system that does not include a locking tube is shown. The needle delivery system includes a delivery needle 210 and an anvil 240. A plurality of suture anchors 220a and 220b (e.g., the suture anchors 300C depicted in FIG. 3G) may be disposed, e.g., end to end, within a central lumen of the delivery needle 210. The anvil 240 is structured and arranged to mechanically interfere with the cantilevered tabs 521 of each suture anchor 220a, 220b and to prevent the anchors 220a, 220b from slipping out of the open end of the delivery needle 210. In some applications, the anvil 240 is cylindrical or substantially cylindrical in shape and has an outer diameter that is sized so as not to mechanically interfere with the side walls 524 (see FIG. 3G) of the anchor 300C. More specifically, when the distal-most suture anchor 220a is urged distally, e.g., by a pushrod 250, as shown in FIG. 2G, the anvil 240 bends or deforms the cantilevered tabs 521 radially inward into a portion of suture, such that the bent or deformed tabs 521 lock the suture to the anchor 220a.

In some implementations, the anvil 240 and anchor 220a are further sized such that advancing the distal-most anchor 220a one anchor length places a junction point 599 between adjacent suture anchors 220a, 220b within a needle bevel 299 of the delivery needle 210. As shown in FIG. 2H, once suture has been locked to the distal-most anchor 220a and the anchor 220a has been deployed, the deployed anchor 220a may be rotated out of the longitudinal axis of the system, e.g., by applying tension to either end of the suture. By applying tension to suture, the deployed anchor 220a rotates about the junction point 599 within the needle bevel 299.

In some surgical procedures, a plurality, e.g., a pair, of locked suture anchors connected by a fixed, predetermined length of suture may be preferred. Those or ordinary skill in the art will appreciate that such an anchored suture system may be prefabricated in lots having specific lengths or may be produced individually at the time of and as part of the surgical procedure.

Referring to FIG. 2I, an embodiment of an anchor delivery system for such an application is shown. The delivery system includes a delivery needle 210 and a pushrod 250. Disposed within the longitudinal, central lumen of the needle 210 is a pair of locked suture anchors 270, 280 that are connected to each other by a fixed length of suture 275. The distal 270 and the proximal anchors 280 of the pair may also be further mechanically coupled to each other, for example, using a frangible connector, e.g., of a type discussed in greater detail above.

Although only a single pair of suture anchors 270, 280 is shown in FIG. 2I, this is done for the purpose of illustration and not limitation. Multiple pairs of anchors may be included, e.g., in a queue, within the delivery needle 210. In some implementations, when there are multiple pairs of anchors 270, 280 within a single delivery needle 210, adjacent anchor pairs may be coupled, for example, using a frangible connector 288, e.g., of a type discussed in greater detail above, such that the coupled anchor pairs may be advanced or withdrawn as a single, more controllable unit. The proximal pair of anchors may also be coupled to the pushrod 250 by a frangible connector 288.

FIG. 2J shows a pair of anchors 270, 280 approximating tissue. In some applications, a first anchor 270 is delivered through a pair of tissue walls 805a, 805b and deployed into a distal lumen, while a second anchor 280 is deployed in a proximal lumen. The fixed length suture 275 connecting the pair of anchors 270, 280 is sized such that once the second anchor 280 is deployed, tension in the suture 275 causes the anchors 270, 280 to compress the tissue walls 805a, 805b together.

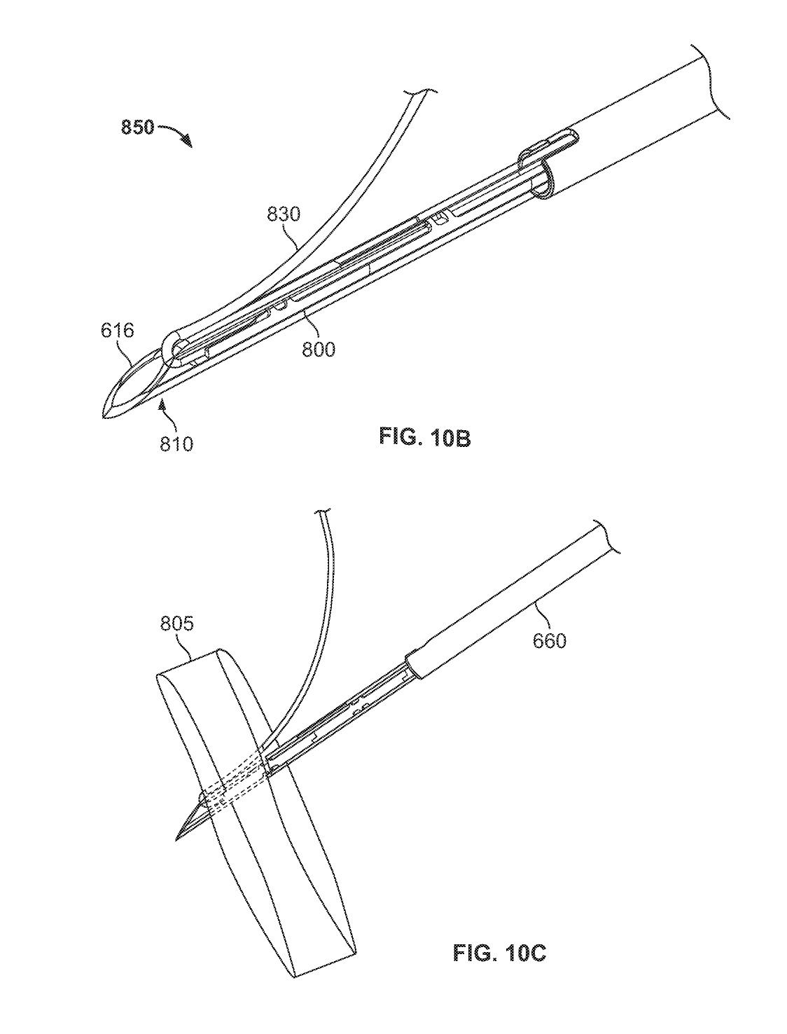

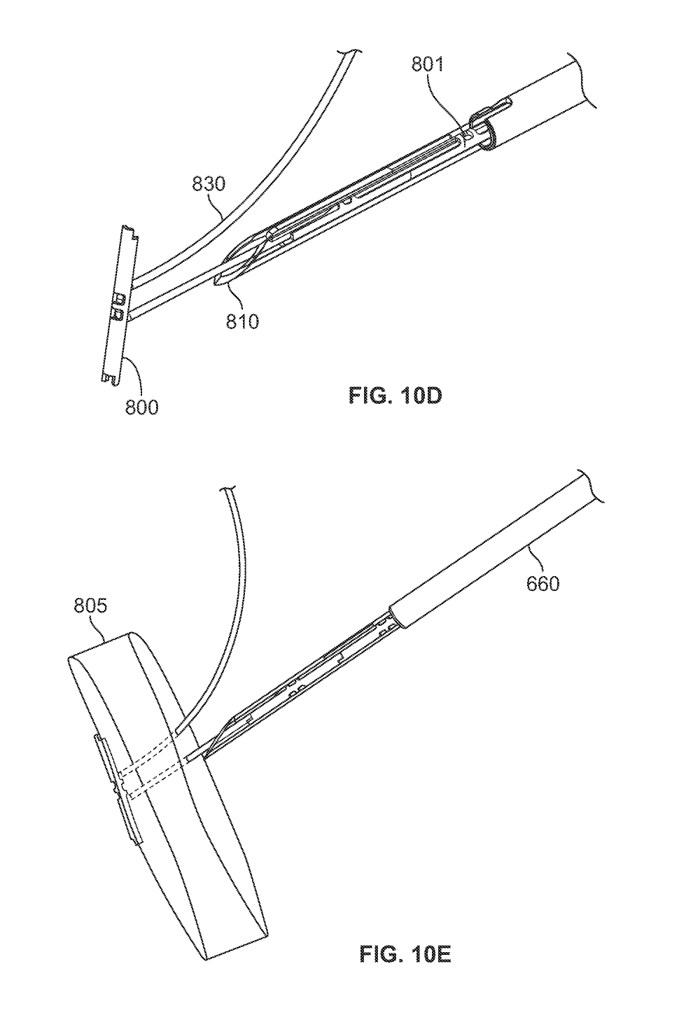

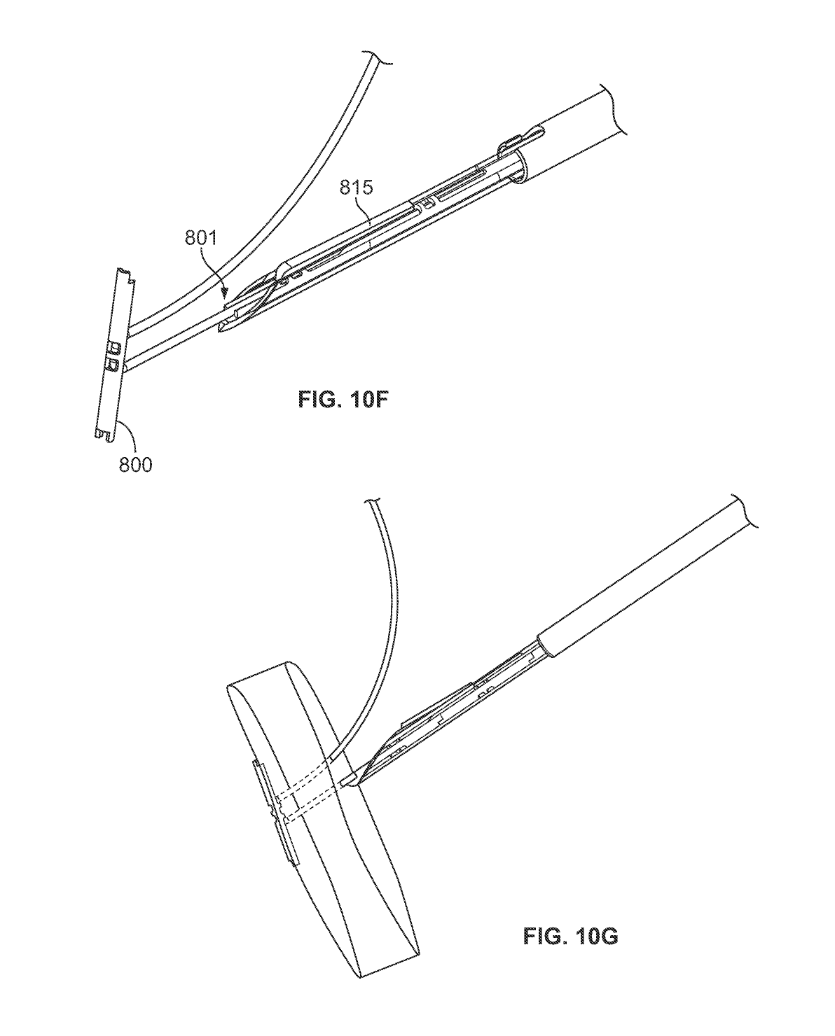

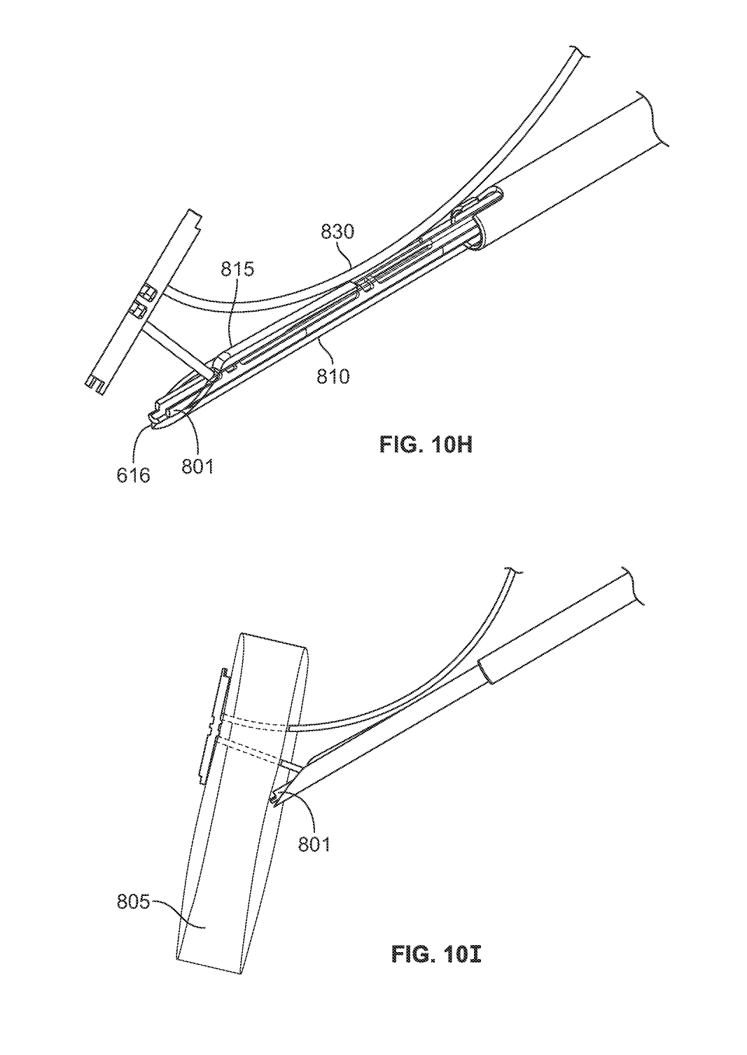

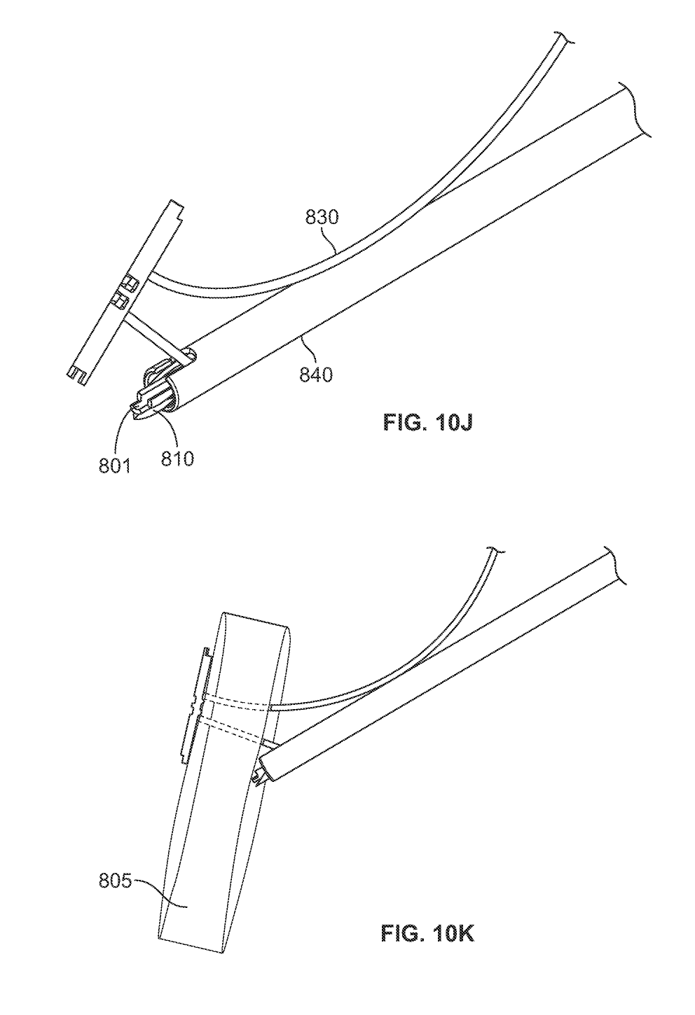

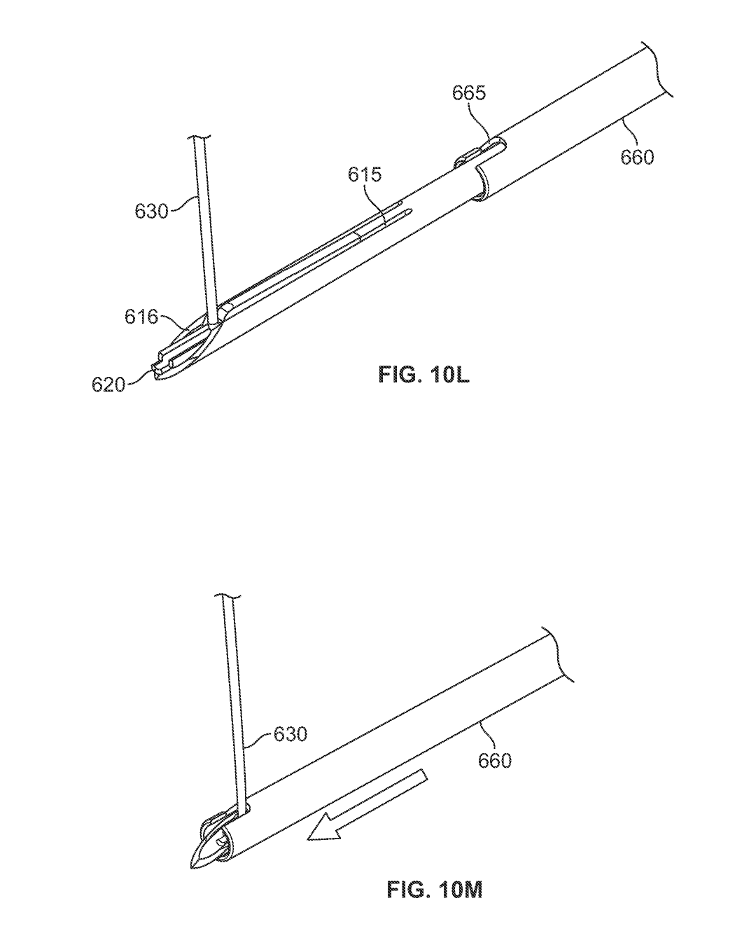

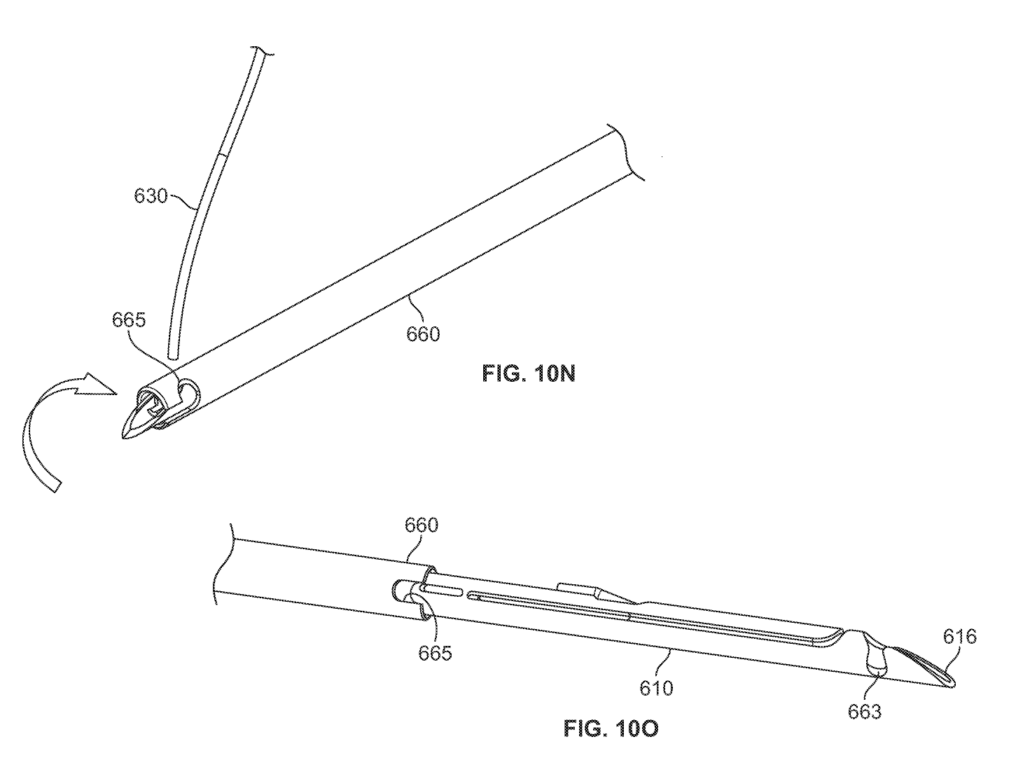

The features of the delivery system and their functions will now be described in connection with describing illustrative embodiments of methods for delivering a suture anchor and suture in a mammalian subject as part of a surgical procedure. Referring to FIGS. 10A and 10B, tissue penetration, anchor delivery, and anchor deployment may be performed either with suture 630, 830 locked in a suture anchor 620, 800 prior to delivery/deployment of the anchor 620, 800 or with suture 630, 830 unlocked in the suture anchor 620, 800. Tissue 805 (FIG. 10C) through which an anchor 620, 800 may be delivered may include relatively soft tissues, such as portions of the GI tract (e.g., the stomach and intestines), layers of muscle (e.g., abdominal muscles), tendon, ligament, cartilage, and so forth. Alternately, tissue 805 may be hard tissue, e.g., cortical bone, and the suture anchor 620, 800 may be configured to deploy within relatively softer tissue, e.g., cancellous bone. Embodiments of the delivery needle 610, 810 configured for delivering an anchor 620, 800 past a layer of hard tissue 805 through a pre-drilled hole may include an unsharpened tip with a distal portion protruding into the needle lumen, such that the anchor 620, 800 is forced radially outwards from the lumen upon delivery. Accessing the tissue site 805 may involve advancing the delivery needle 610, 810 through an instrument channel of an endoscope.

FIG. 10A depicts an illustrative embodiment of the former instance, in which a suture anchor delivery system 600, including a delivery needle 610, is configured for delivering a locked suture anchor 620 longitudinally through tissue 805. The suture anchor 620 is shown in an enclosed, locked position in which one or more anchor bridges are under the anvil 640 while the body of the anchor 620 remains entirely within the lumen of the delivery needle 610, in preparation for locking to suture 630. As previously described, the delivery needle 610 may include anvil slots 615 within which the anvil 640 may be supported at the end of the cantilever 616. In some variations, the anvil 640 may be disposed proximally of the needle tip, e.g., the needle bevel 616, leaving a distal space in the slot 615 into which suture 630 may fold, aiding tissue penetration and providing lateral support for suture cutting (described below). As shown in FIG. 2D, before penetrating tissue 805, the locking tube 260 may be translated distally over the delivery needle 210 to press the anvil 240 into the bridge(s) 221 of the anchor 220a, locking the suture anchor 220a to suture 230 (STEP 1).

Alternatively, FIG. 10B shows an illustrative embodiment of the latter instance, in which a suture anchor delivery system 850, including a delivery needle 810, is configured for delivering an enclosed, unlocked suture anchor 800 through tissue 805. As shown in FIG. 10B, when delivering an unlocked suture anchor 800, the suture anchor 800 remains entirely within the lumen of the delivery needle 810 and some distance away from the opening at the needle bevel 616. In some variations, the anvil 640 may be disposed proximally of the needle tip, e.g., the needle bevel 616, leaving a distal space in the slot 615 into which suture 830 may fold, aiding tissue penetration and providing lateral support for suture cutting.

Delivery and deployment of one or more suture anchors 620, 800, whether with or without suture locked in the suture anchor, are shown illustratively in FIGS. 10C through 10G. First, the delivery needle 610, 810 may be inserted through tissue 805 (STEP 2) to a desired depth. In some implementations, during delivery (STEP 2), suture 630, 830 may extend proximally within the anchor 620, 800 as well as distally outside of the delivery needle 610, 810. Once the delivery needle 610, 810 has been advanced to a desired depth (STEP 2), a first suture anchor 620, 800 may be urged out of the lumen of the delivery needle 610, 810, e.g., by urging the suture anchor 620, 800 with the pushrod 250, and deployed behind the tissue layer 805 (STEP 3) (FIG. 10D). In some applications, the suture anchor 620, 800 may be deployed within a portion of thicker tissue 805.

Once a first or any subsequent suture anchor 620, 800 has been deployed (STEP 3) (FIG. 10D), as shown in FIG. 10E, the delivery needle 610, 810 may be withdrawn from behind the tissue 805 (STEP 4). Those of ordinary skill in the art will appreciate that while the first suture anchor 620, 800 is being deployed, especially when urged by the pushrod 250, a second suture anchor 801 is displaced to the position the first suture anchor 620, 800 was at prior to the optional locking operation (STEP 1) and the delivery operation (STEP 2) having been performed. Accordingly, after withdrawal of the device from behind the tissue 805, as shown in FIGS. 10F and 10G, a second suture anchor 801 may already overlap the sharp portion of the needle 610, 810 and the anchor bridge(s) of the second suture anchor 801 may already mechanically interfere with the anvil 815, forcing the anvil 815 radially outwards. If, for some reason, the device was not originally filled with multiple suture anchors or a suture anchor had to be subsequently added, the second or subsequent suture anchor 801 may be advanced within the lumen of the delivery needle 610, 810 until the suture anchor 801 overlaps the sharp portion of the needle 610, 810 and the anchor bridge(s) of the suture anchor 801 mechanically interferes with the anvil 815, forcing the anvil 815 radially outwards. At this point, the suture 630, 830 may or may not be locked into the advanced, suture anchor 801, which is ready to be delivered and deployed in the same manner as described in connection with STEPS 2 through 4.

In some implementations, a selectively-lockable suture anchor(s) 620, 800 may be augmented with one or more suture locks 700, which may resemble suture anchors 620, 800 but without lateral tissue supports. Referring to FIGS. 11A and 11B, a suture lock 700 may include locking features such as a deformable bridge(s) 703 and one or more suture pockets 704, toothed slots, or any combination of suture locking features as previously described concerning the various anchor configurations. The suture lock 700 may also form a lumen 705. The anchor bridge(s) 703 and lumen 705 provide a passage and mechanical clearance through which suture may freely and slidably travel bi-directionally. Advantageously, the bridge(s) 703 may be configured to slidably accept suture in an unlocked state and be affixed to the suture in a locked state. The suture lock 700 may also include male 701 and female 702 anti-rotation keys configured to mate with corresponding anti-rotation features integrated into a suture anchor 620, 800 and/or the pushrod 250.

The suture lock 700 may be delivered and deployed in a similar manner as a locked suture anchor 620. For example, in some applications, after an unlocked suture anchor 800 has been delivered and deployed on a distal side of tissue 805, the suture lock 700 may be deployed on the same side of the tissue 805, after suture 730 has been locked in the suture lock 700. Advantageously, combining a suture lock 700 with an unlocked suture anchor 800 on the same, e.g., distal, side of a tissue layer 805 enables a surgeon or other medical personnel to deploy the unlocked suture anchor 800 first—in which suture 730 is free to move—then to tension suture 730 before the suture lock 700 and suture 730 are affixed to one another. As shown in FIG. 11B, a series of suture anchors 720a, 720b and suture locks 700 may be contained, e.g., in a queue, within a delivery needle 710 with a coaxial portion of suture 730. Although FIG. 11B shows a suture lock 700 disposed between a first 720a and a second suture anchor 720b, this is done for illustrative purposes only. Advantageously, the surgeon using the device may include any number of, including zero, suture locks 700 between suture anchors 720a, 720b, as the medical procedure dictates.

In some applications, the nature of the procedure may require suture tensioning, which may require, for example, drawing tissue 805 toward the flat surface of the needle bevel 616. As shown in FIGS. 10H through 10K, the suture 830 may be tensioned by retracting suture 830 proximally through the delivery needle 810 so as to bring tissue layer 805 into contact with the second suture anchor 801 (FIG. 10I). As shown in FIGS. 10J and 10K, the second suture anchor 801 may be placed in a more distal position within the delivery needle 810 such that the distal portion of the anchor 800 overlaps the sharp portion of the delivery needle 810 while the anchors' bridge remains under the anvil 815. In this configuration, the suture 830 may be withdrawn through the needle 810 and anchor 801 to tension a stitch while tissue 805 is simultaneously being pulled against the needle bevel 616 that is protected by the overlapping portion of anchor 801. Once the appropriate amount of tension has been added, the locking tube 840 may be advanced distally over the delivery needle 810, mechanically interfering with anvil 815 and forcing it radially inwards, thereby locking the second suture anchor 801 to suture 830. The second suture anchor 801 may then be deployed in the same manner as described in connection with STEPS 2 through 4.

Once the desired number of anchors has been deployed, the suture—as the procedure may dictate—may be cut (STEP 5). Referring to FIGS. 10L through 10N, the locking tube 660 may include a recessed cutting edge 665 that enables it to cut suture 630 (STEP 5). For example, in some embodiments, cutting edge 665 may be configured for cutting suture 630 in a circumferential direction when the locking tube 660 is advanced distally then rotated about its longitudinal axis. In alternative embodiments, cutting edge 665 may be configured for cutting suture 630 along the longitudinal direction, or both in the longitudinal and circumferential directions. Suture 630 may protrude from the anvil slot 615 during cutting for circumferential support or it may protrude from the needle bevel 616 and be supported by a portion of the bevel 616. Locking an anchor 620 in the delivery needle and then cutting suture 630 is advantageous when finishing a stitch and preparing to start another.

An alternative suture-cutting structure is shown in FIG. 10O. With this embodiment, the locking tube 660 may include a cutting edge 665 that enables it to cut suture 630 disposed in a suture-cutting notch 663 of the delivery needle 610 (STEP 5). Advantageously, the cutting edge 665 is configured for cutting suture 630, without rotation, in a longitudinal direction when the locking tube 660 is advanced distally. More specifically, suture 630 may protrude from a suture-cutting notch 663 formed in the delivery needle 610 and/or in the needle bevel 616. The suture-cutting notch 663 may also be sharp to aid in cutting the suture 630.

The embodiments of the suture anchor and delivery system detailed in FIGS. 10A through 10O may be implemented in a longer, more flexible format suitable for flexible endoscopy applications within the scope of the present invention. A shorter, rigid system may be employed advantageously in procedures with more direct access, such as various laparoscopic procedures.

Illustrative uses of the device will now be described. Those of ordinary skill in the art will appreciate that the exemplary uses are for the purpose of illustration and not limitation. Those skilled in the art will also appreciate the myriad of applications of the device. Advantageously, the device may be used for both interrupted and running suture placements, permitting the same device to place one or more interrupted or running sutures.

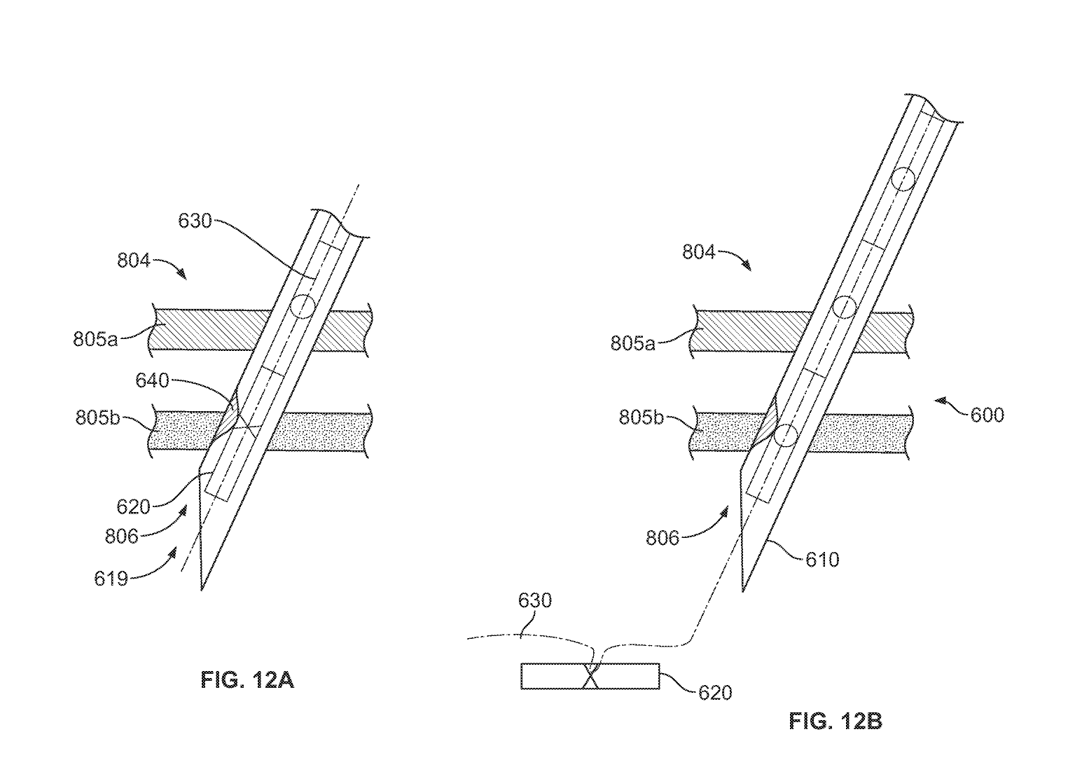

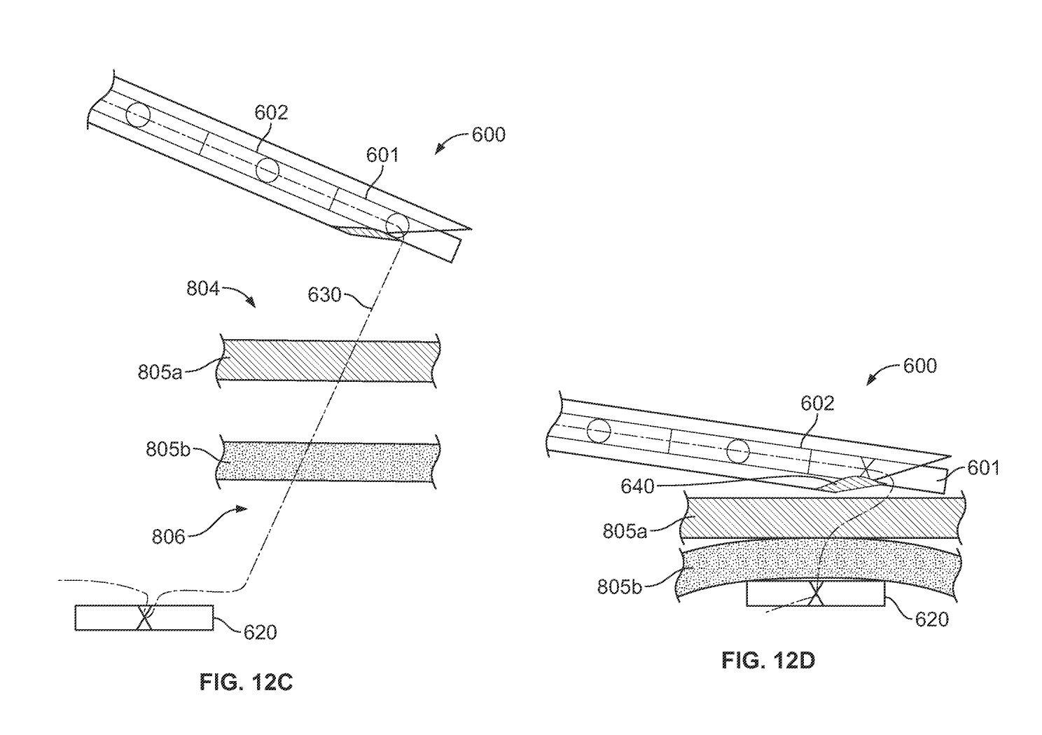

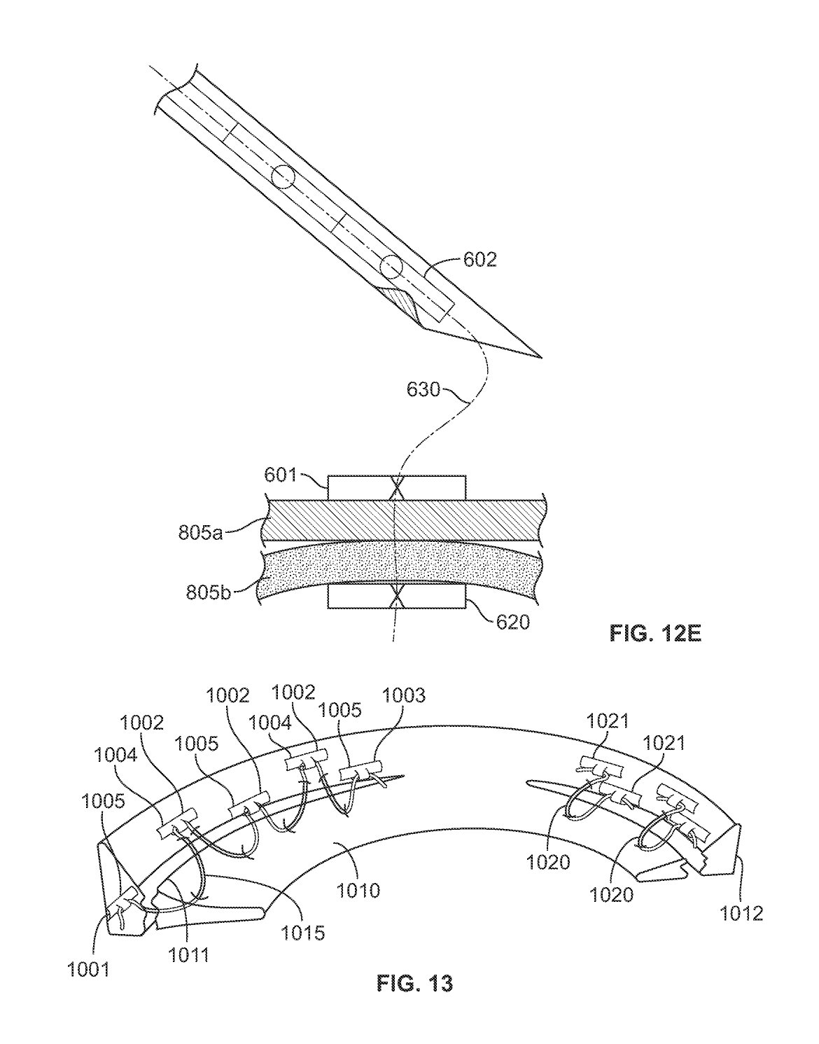

Using the needle delivery system 600 depicted in FIG. 10A, a first exemplary use for approximating tissue planes is depicted in FIGS. 12A through 12E. From a proximal lumen 804 in body structure, the needle delivery system 600 is punctured into a second, distal lumen 806 in body structure, e.g., from the stomach into the jejunum. After or before puncture across the desired tissue planes of the lumen walls 805a and 805b, a suture locking structure associated with the distal-most suture anchor 620 within the delivery system 600 may be selectively engaged, i.e., locked, e.g., by an anvil 640 located towards the distal end 619 of the delivery needle 610 (STEPS 1 and 2) (FIG. 12A). This locks suture 630 to this specific suture anchor 620. The suture anchor 620, with locked suture, is then advanced, i.e., deployed, out of the needle delivery system 600 (STEP 3) (FIG. 12B). The needle delivery system 600 may then be withdrawn back to the proximal lumen 804 (STEP 4) (FIG. 12C), leaving the distal suture anchor 620 in the second, distal lumen 806. The suture 630 that runs along the length of the non-deployed anchors 601, 602 within the delivery system 600 is untensioned and free to extend out of the delivery system 600 during needle-tip withdrawal.

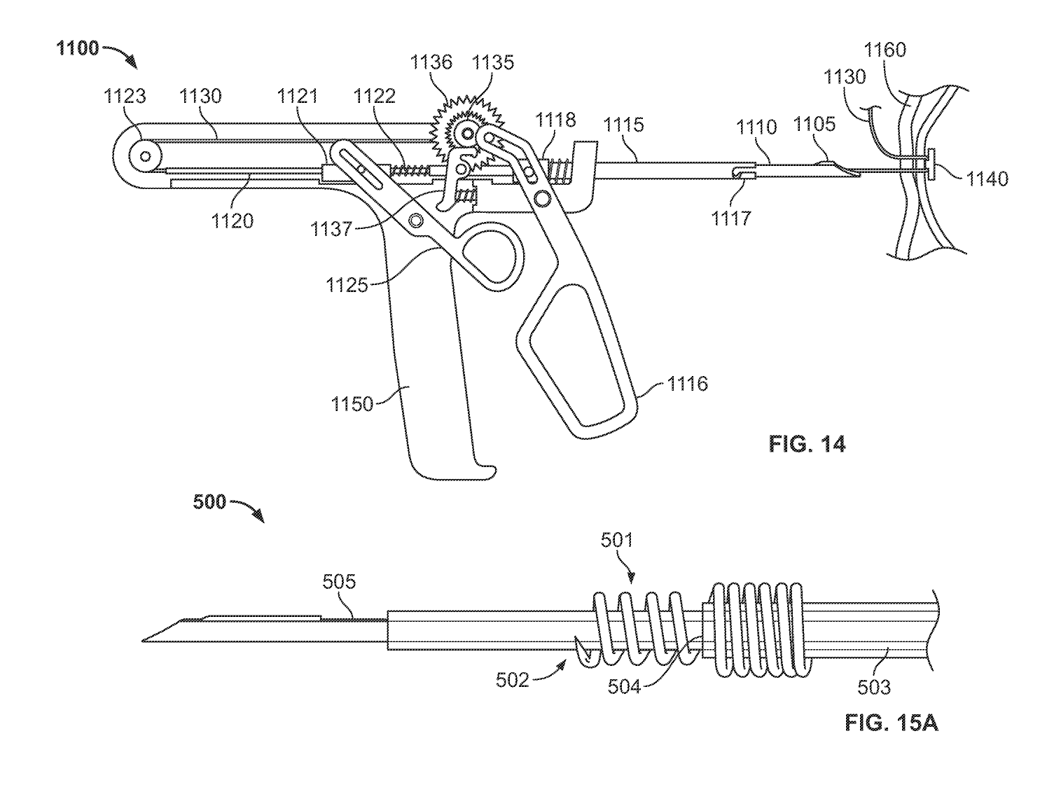

The next-in-sequence suture anchor 601 that is in the delivery system 600 may then be advanced towards the distal tip 619 of the delivery needle 610 in such a position that the distal end of this anchor 601 extends to cover the sharp edge of the needle tip without being completely advanced out of the delivery system 600 (FIG. 12C). In such a position, tension can be applied to the proximal aspect of the slidable suture 630 with some forward or stable force to the delivery system 600 to approximate the two lumen walls 805a, 805b (FIG. 12D) without causing needle-tip related trauma. Tension on the suture 630 may be applied manually by the operator, or via mechanical refraction mechanisms, e.g., a constant force spring mounted spool, an electric motor under feedback control, a manually-operated suture reel attached to the handle of the delivery system (FIG. 14), or any suitable suture retraction mechanism.

Once the suture 630 has been tightened sufficiently so that the two lumen walls 805a, 805b are in desired proximity, the suture locking structure on or near the next-in-sequence suture anchor 601 may be engaged, e.g., by an anvil 640 located towards the distal end of the delivery system 600 (FIG. 12D). This locking mechanism may lock the suture 630 to this second anchor 601 in or near the proximal lumen wall 805a, thereby maintaining suture tension and apposition of the tissue between the two lumens 804, 806. The second anchor 601 may then be deployed and the suture 630 cut (STEP 5) proximal to the locked position near the second suture anchor 601. A new suture placement using the next-in-sequence, selectively-lockable suture anchor 602 may then be started using the same steps as described above.

Alternatively, another interrupted suture may be initiated without cutting of the suture 630 as shown in FIG. 12E. Advantageously, this procedure may allow for looped formations of the suture 630, between separate cinched and locked suture anchors that approximate the two lumen walls 805a, 805b at multiple areas. Tension may then be applied to such a looped suture formation to assist in advancing, for example, a trocar or other such device from the proximal lumen 804 to distal lumen 806 through the approximated lumen walls 805a, 805b.

A second use of the needle delivery system 600 may involve an interrupted suture for use, for example, in closing a tissue defect or perforation. In some implementations, the needle delivery system 600 is punctured across a tissue plane on one side of a defect. A suture locking structure of the distal suture anchor 620 is engaged with an anvil 640 towards the distal end 619 of the delivery needle 610. The locking mechanism may lock suture 630 to this specific suture anchor 620. The anchor 620, with locked suture 630, may then be advanced out of the delivery needle 610 and deployed at a desired location. The needle 610 may then be withdrawn from the tissue plane, leaving the anchor 620 with locked suture 630 across the puncture site and at the distal aspect of the tissue plane. The delivery needle 610 may then be punctured across a tissue plane on an opposing side of the defect. The next in-sequence (second) anchor 601 may then be advanced out of the needle delivery system 600 without engaging the suture locking structure, i.e., the anchor 601 remains unlocked. The delivery needle 610 may then be withdrawn, leaving this unlocked anchor 601 with a slidable suture 630 at the distal aspect of the tissue plane and on an opposing side of the defect.

The next in-sequence (third) suture anchor 602 in the delivery system 600 may then be advanced towards the distal tip of the delivery needle 610 so that the distal end of this anchor 602 extends to cover the sharp edge of the needle tip without being completely advanced out of the delivery system 600. In such a position, tension can be applied to the proximal aspect of the slidable suture 630 to approximate the two aspects of the defect without causing needle-tip related trauma. The suture locking structure may then be engaged, e.g., by an anvil 640 located towards the distal end 619 of the delivery system 600. This may lock the suture 630 to this anchor 602, and thereby maintain suture tension between anchors 620, 601, and 602 and bring together two aspects of a tissue defect or perforation. The third anchor 602 may then be deployed and the suture 630 cut proximal to the locked position of this (third) anchor 602. A new suture 630 placement may then be initiated.

A specific application of the above method involves the arthroscopic repair of torn meniscus cartilage. As shown in FIG. 13, suture anchors 1004, 1005 may be deployed in a vertical zigzag configuration, respectively deployed above and through a tear 1011 in a meniscus cartilage 1010, to form a running z-stitch repair using running suture 1015. The series of anchors 1004, 1005 may be deployed through the cartilage such that they rest on the outer surface 1012 of the cartilage with the initial deployed anchor 1001 locked and intermediate deployed anchors 1002 unlocked such that the z-stitch may be tightened prior to deploying the terminating anchor 1003. Alternatively, each anchor may be locked and the suture may be tightened as each anchor is deployed.

In an alternative suture configuration, suture anchors 1021 may be deployed in a series of interrupted stitches 1020 with both anchors 1021, e.g., anchors above and through the tear, locked. With both of these suture configurations, the cartilage may be approached from the meniscal surface so as leave only suture on the meniscal surface 1012 and to endoscopically confirm complete closure of the tear.

In another exemplary embodiment, the needle delivery system 600 depicted in FIG. 10A facilitates the use of a running suture. The suture locking structure may be engaged with an anvil 640 towards the distal end of the needle delivery system 600. This locking mechanism locks the suture 630 to the distal most anchor 620. The needle delivery system 600 may then puncture across one or more tissue planes 805. The anchor 620, with locked suture 630, may then be advanced, i.e., deployed, out of the delivery needle 610 to serve as the first anchor point for the suture 630. The delivery needle 610 may then be withdrawn, leaving the anchor 620 with locked suture 630 across the punctured tissue planes.

The needle delivery system 600 may then puncture across another aspect of the tissue planes. A next in-sequence anchor 601 is advanced out of the delivery needle 610 without engaging the suture locking mechanism, i.e., unlocked. The delivery needle 610 may then be withdrawn, leaving this anchor 601 with a slidable suture 630 at the distal aspect of the tissue planes and across the second puncture site. Additional punctures across various aspects of tissue planes may be made, as deemed necessary, in the same manner. Punctures with placement of suture anchors with a running slidable suture 630 can be made in purse-string, crisscross, z-shaped, or any similar pattern as desired.

When the desired length and pattern of running suture is complete, a final anchor can be deployed. In some applications, this may be done with the needle delivery system 600 located at the proximal aspect of the tissue planes. The next in-sequence (final) anchor may be advanced towards the distal tip of the delivery needle 610 in such a position that the distal end of this anchor extends to cover the sharp edge of the needle tip without being completely advanced out of the delivery system 600. In such a position, tension can be applied to the proximal aspect of the slidable suture 630 to cinch the running suture 630 between suture anchors without causing needle-tip related trauma. The suture locking structure of the final anchor may then be engaged with an anvil 640 located towards the distal end of the delivery system 600. This mechanism locks the suture 630 to the final anchor, thereby maintaining suture tension between anchors and keeping the tissue planes, which have been pierced with the multiple suture anchors, together. The final anchor may then be deployed and the suture 630 cut proximal to the locked position of the final anchor. A new suture 630 placement may then be initiated.

Another application of the present invention may include alternating suture anchors and suture locks. For example, with reference to FIG. 11B, in some embodiments the needle delivery system features an alternating series of unlocked suture anchors 720a, 720b and suture locks 700, with a single suture lock 700 following and between each suture anchor 720a, 720b. In use, suture locks 700 may be affixed to suture 730 or may be left slidably attached over the suture 730 if locking is not desired. While more complex than embodiments employing only lockable suture anchors, this configuration may be advantageous in enabling the terminating suture anchor to fully exit the delivery needle 710 before the suture 730 is tensioned and a suture lock 700 is affixed to suture 730. Moreover, suture anchors 720a, 720b under tension may be placed at will at either the proximal or distal surface of tissue 805 being sutured.

FIG. 14 depicts an embodiment of a handheld, manually-actuatable suture anchor delivery system 1100 in which suture 1130 may extend slidably through the respective lumens of a series of suture anchors 1140, optional suture locks, and pushrod 1120 contained at least partially within the delivery needle 1110. In some variations, drag friction between a suture reel 1135 and supporting handle 1150 may be desired, for example, to prevent the suture reel 1135 from spinning freely when suture 1130 is not being actively pulled. Suture 1130 extending through the delivery needle 1110, suture anchors 1140, optional suture locks, and pushrod 1120 may be retracted and/or tensioned by rotating, e.g., counterclockwise, as shown, the suture reel 1135 that serves as a reservoir of available suture 1130. The suture reel 1135 may include a knurled thumbwheel 1136 configured for easy access by the thumb of the user's hand supporting the delivery system 1100, especially for rotation of the reel 1135 to retract and/or tension the suture 1130.

In some implementations, tension on the suture 1130 may be maintained by a spring-loaded, ratcheting reel lock 1137, a portion of which may act as a pawl to engage tooth features in the suture reel 1135. During use, in some applications, tension in suture 1130 may be released by depressing the lever portion of the reel lock 1137 to disengage the pawl portion of the reel lock 1137 from suture wheel teeth. Advantageously, this allows the user to release loose suture 1130 from the distal end of the delivery needle 1110 when withdrawing the delivery system 1100.

During use, suture anchors 1140 and/or suture locks may be advanced distally within the delivery needle 1110 by actuating an anchor advance lever 1125, e.g., a finger pull loop, that is structured and arranged to advance or propel a pushrod slider 1121 and a pushrod pawl 1122 towards the distal end of the delivery system 1100. In some embodiments, the pushrod slider 1121 is operationally coupled to the pushrod 1120 by the pawl 1122, e.g., by engaging longitudinally-spaced tooth features on the pushrod 1120. In some variations, the pushrod 1120 may be enclosed in a portion of the handle 1150, to protect the pushrod 1120 during use and handling.

In some implementations, the handle 1150 may also include a suture guide 1123 that is structured and arranged to direct the suture 1130 out of the pushrod 1120 and to the suture reel 1135, which may be located distally to the proximal end of the pushrod 1120.

Once the distal-most suture anchor 1140 within the delivery needle 1110 has advanced sufficiently distally to force the anvil 1105 radially outwards, the suture anchor 1140 may be locked to the suture 1130, e.g., by advancing distally a locking tube 1115 actuated by an anchor locking lever 1116. In some variations, actuation of the locking lever 1116 advances or propels distally a locking tube slider 1118 that is coupled to the locking tube 1115 such that, as described in greater detail hereinabove, the locking tube 1115 forces the anvil 1105 radially inwards to lock suture 1130 within the distal-most anchor within the delivery needle 1110. In a second stage of anchor locking lever actuation, which may be demarcated by tactile feedback, the locking tube 1115 may be rotated on its axis such that a cutting edge 1117 presses into and bisects suture 1130. Other embodiments of the invention may include robotic actuation in place of each of the manually actuated elements. Feedback control of the suture reel, and haptic feedback in particular, may be advantageous in robotic embodiments of the delivery system.

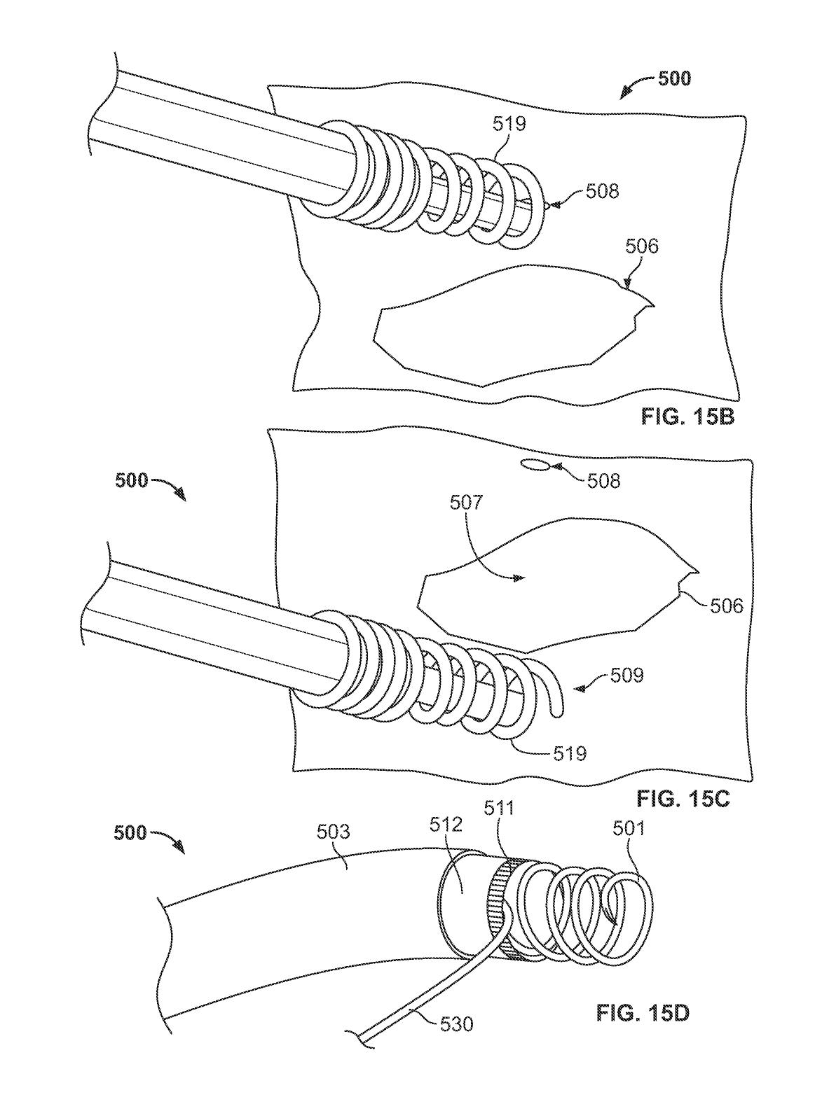

Referring to FIGS. 15A through 15C, a helical tissue control tool 500 for tissue retraction and control in connection with the needle delivery system(s) described above is shown. In some embodiments, the tissue control tool 500 includes a helical corkscrew element 501, having a sharpened distal tip 502, that is affixed to a catheter 503 having a central lumen 504, through which an instrument 505, e.g., a needle delivery system 600, may be inserted and withdrawn. Such instruments 505 may also include tissue aspiration needles, biopsy forceps, injection needles, tissue approximation devices, and cryotherapy or RF ablation probes. In some variations, the helical tip 502 may be configured to engage a tissue plane with a rotational force or torque applied, e.g., to the catheter 503, at a proximal end, and to disengage the tissue with an opposite rotational movement.

The shaft of the device 500 may be composed of rigid (for laparoscopic) or flexible (for endoscopic) material that may be made of metal, polymer, fiber-reinforced composite, or other suitable materials. As an example of use with a suture anchor tissue apposition tool 510 as shown in FIG. 15B, the tip 502 of the helical corkscrew element 501 may be pressed against a first target site 508 and rotated, e.g., clockwise, to penetrate into one edge of a tissue defect 506. With the tissue supported by the helical corkscrew element 501, a medical instrument, e.g., a tissue apposition tool 510, may thereafter be advanced through the central lumen 504 and through the supported tissue layer. Using techniques and methods previously described, one or more suture anchors may then be delivered and deployed, selectively with or without suture locked in the anchor, on the distal side of a tissue layer before the helical corkscrew element 501 is rotated out, e.g., counterclockwise, from the tissue.

As shown in FIG. 15C, the device 500 and tissue apposition tool 510, to which suture 507 is still attached, may then be relocated to a second target site 509 on an opposite edge of the tissue defect 506. With suture 507 extending from the first deployed suture anchor(s), one or more additional suture anchor(s) or locks may be delivered and deployed to a desired location within the tissue.

Referring to FIG. 15D, an alternative embodiment of the tissue control tool 500 is shown. A cutting edge 511 is integrated into the tissue control tool 500, such that the tool 500 may both control the tissue during needle penetration and sever suture 530, e.g., after one or more suture anchors have been deployed. In some implementations, the cutting edge 511 is formed on a portion of a, e.g., metallic, base 512, to which the helical corkscrew element 501 is affixed. In some variations, the cutting edge 511 is beveled radially inwards, so that the cutting edge 511 is shielded from the helical corkscrew element 501.

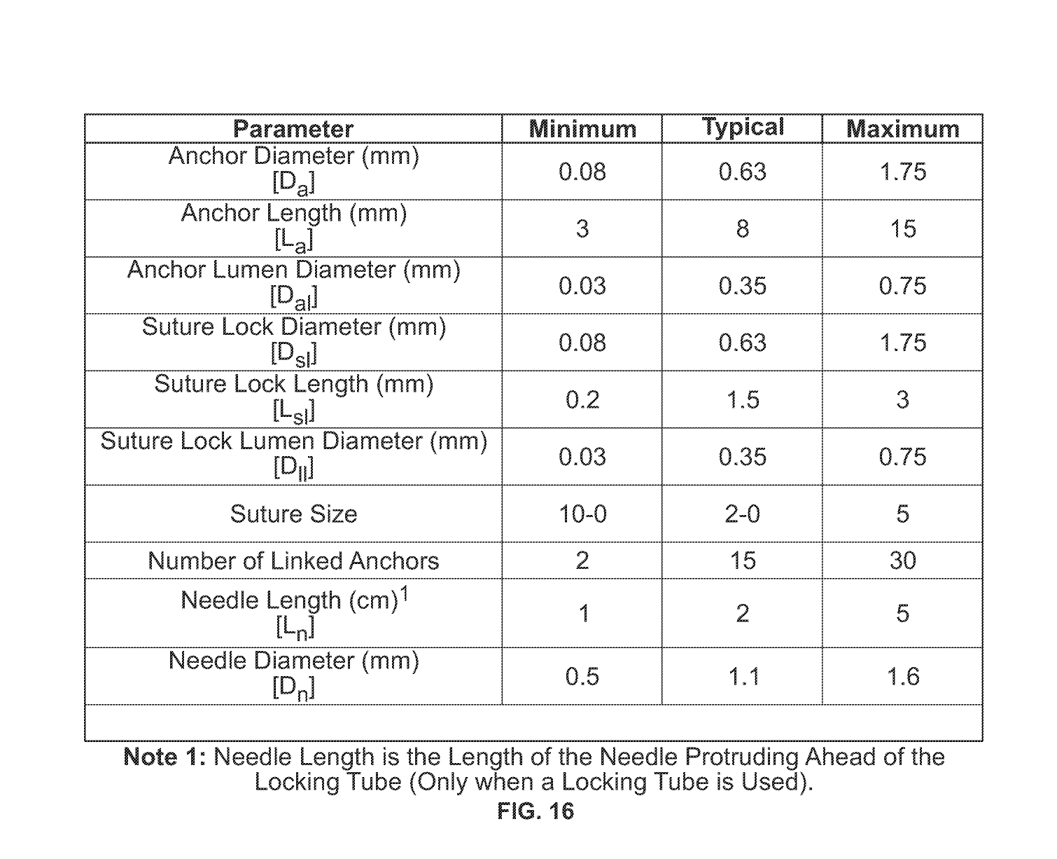

FIG. 16 depicts a table of geometric and other parameters for various embodiments of anchors, suture locks, suture, and delivery needles. The quantities and values should be considered to be illustrative of typical embodiments, but not necessarily limiting. Various applications may require larger or smaller components and delivery systems and are within the scope of the invention.

Those of ordinary skill in the art will realize that the invention may be embodied in other specific forms without departing from the spirit or essential characteristics thereof. The foregoing embodiments, together with the attached drawings, are, therefore, to be considered in all respects illustrative rather than limiting of the invention described herein.

What is claimed is:

1. A suture anchoring system, comprising:
an elongate anchor comprising a longitudinal axis extending from a proximal end to a distal end of the anchor and a displaceable section for locking a portion of suture so that a length of the locked suture portion extends in a direction of the longitudinal axis.

2. The system of claim 1, wherein the displaceable section comprises at least one of a plastically deformable material and an elastically deformable material.

3. The system of claim 1, wherein the displaceable section comprises a structure selected from the group consisting of a suture loop, a suture locking tab, a deformable connecting element, a suture engagement slot, a suture engagement tooth, a deflectable locking prong, a bridge element, a deformable strut, and a cantilevered tab.

4. The system of claim 1, wherein the anchor further comprises a fixed section comprising at least one feature for engaging the suture.

5. The system of claim 4, wherein the at least one feature comprises a friction-enhancing feature selected from the group consisting of a suture engagement slot, a suture engagement tooth, and a fixed tab.

6. The system of claim 1, wherein the anchor further comprises a plurality of tissue supports.

7. The system of claim 1, wherein the anchor further comprises an anti-rotation key mateable with a corresponding anti-rotation key of another anchor.

8. The system of claim 1, wherein the anchor further comprises a beveled distal tip and a connector interlockable with a corresponding connector of another anchor to permit rotation of the anchor in a single plane.

9. The system of claim 1, wherein the anchor further comprises at least one of a sharp distal tip and a blunt shoulder proximate the distal tip.

10. The system of claim 1, wherein the anchor defines a lumen for slideably accepting the suture.

11. The system of claim 1, wherein the anchor defines at least one suture pocket for accepting a portion of the suture.

12. The system of claim 1 further comprising the suture.

13. The system of claim 12, wherein at least one of the anchor and the suture comprises a bio-resorbable material.

14. A system for delivering suture anchors, comprising:
a delivery needle; and
an anvil coupled to the delivery needle for locking, within the delivery needle, an anchor to a suture.

15. The system of claim 14 further comprising:
a plurality of anchors positioned within the delivery needle; and
the suture extending slideably through the plurality of anchors.

16. The system of claim 15, wherein the delivery needle, each anchor, and the suture are aligned substantially coaxially.

17. The system of claim 15 further comprising a frangible connector between each anchor.

18. The system of claim 17, wherein each anchor comprises disengagement shoulders at proximal and distal ends of the anchor.

19. The system of claim 15 further comprising a pushrod for advancing the plurality of anchors through the delivery needle.

20. The system of claim 19, wherein the pushrod comprises an anti-rotation key mateable with a corresponding anti-rotation key of an anchor.

21. The system of claim 15 further comprising a mechanism for at least one of retracting, tensioning, and releasing the suture.

22. The system of claim 14 further comprising a locking tube, moveable relative to the delivery needle, for actuating the anvil.

23. The system of claim 22, wherein the locking tube comprises a cutting edge.

24. The system of claim 14, wherein the delivery needle defines a longitudinal slot for engaging a protruding tab of an anchor.

25. The system of claim 14, wherein the delivery needle comprises a sharp distal tip.

26. The system of claim 14, wherein the delivery needle comprises a suture-cutting notch.

27. A method for suturing tissue, the method comprising the steps of:
using an anvil to lock, within a delivery needle, an anchor to a suture; and
deploying the anchor from the delivery needle to a tissue site.

28. The method of claim 27, wherein the step of using the anvil comprises actuating the anvil by displacing a locking tube relative to the anvil.

29. The method of claim 27, wherein the step of using the anvil comprises causing the anvil to mechanically interfere with a displaceable section of the anchor.

30. The method of claim 29, wherein the step of causing the anvil to mechanically interfere with the displaceable section of the anchor comprises displacing the anchor with a pushrod.

31. The method of claim 27 further comprising the step of displacing a locking tube to cut the suture.

32. The method of claim 31, wherein the step of displacing the locking tube to cut the suture comprises at least one of moving the locking tube longitudinally along a longitudinal axis of the delivery needle and rotating the locking tube.

33. The method of claim 27 further comprising the step of advancing the delivery needle through an instrument channel of an endoscope to the tissue site.

34. The method of claim 27 further comprising the step of using the anchor and the suture to perform at least one of an approximation of tissue layers, an approximation of two separate lumens within a patient's body, a closure of a defect at the tissue site, and securing of a medical device at the tissue site.

35. The method of claim 34, wherein the medical device is selected from the group consisting of a stent, a physiological sensor, a camera, and a surgical mesh.

36. A tissue engagement tool, comprising:
a catheter comprising a lumen for receiving a medical instrument;
a cutting edge coupled to a distal end of the catheter; and
a helical corkscrew element coupled to the distal end of the catheter.

37. The tissue engagement tool of claim 36, wherein the cutting edge is beveled radially inwards.

38. The tissue engagement tool of claim 36, wherein the helical corkscrew element comprises a sharp distal tip for engaging tissue.

39. A method for engaging tissue, the method comprising the steps of:
inserting a tissue engagement tool proximate a tissue site, the tissue engagement tool comprising a catheter and a helical corkscrew element coupled to a distal end of the catheter;
rotating the helical corkscrew element to engage the tissue site; and
thereafter delivering a medical instrument through a lumen of the catheter to the tissue site.

40. The method of claim 39 further comprising the step of cutting suture using a cutting edge coupled to the distal end of the catheter.

41. The method of claim 39, wherein the medical instrument is a device selected from the group consisting of a tissue approximation device, a tissue aspiration needle, biopsy forceps, an injection needle, a cryotherapy probe, and an RF ablation probe.
